(19) United States
(12) Patent Application Publication
(10) Pub. No.: US 2003/0148381 A1
(43) Pub. Date: Aug. 7, 2003

(54) More protein-protein interactions in the inner ear

(75) Inventors: Laurent Daviet, Paris (FR); Pierre Legrain, Paris (FR); Christine Petit, Le Plessis Robinson (FR); Batiste Boeda, Paris (FR); Aziz El-Amraoui, Paris (FR)

Correspondence Address: LERNER, DAVID, LITTENBERG,, KRUMHOLZ & MENTLIK, 600 SOUTH AVENUE WEST, WESTFIELD, NJ, 07090, US

(73) Assignee: Hybrigenics, Paris (FR)

(21) Appl. No.: 10/177,191

(22) Filed: Jun. 21, 2002

Related U.S. Application Data
(60) Provisional application No. 60/299,848, filed on Jun. 21, 2001.

(30) Foreign Application Priority Data
Feb. 5, 2002 (EP) 02290277.9

(51) Int. Cl. A61K 31/00; G01N 33/53; C12P 21/02; C12N 5/06; C07K 14/47; C07H 21/04

(52) U.S. Cl. 435/7.1; 435/325; 435/320.1; 530/350; 514/1; 536/23.1

(57) ABSTRACT

The present invention relates to protein-protein interactions involved in deafness or in hearing disorders and/or diseases. More specifically, the present invention relates to complexes of polypeptides or polynucleotides encoding the polypeptides, fragments of the polypeptides, antibodies to the complexes, Selected Interacting Domains (SID®) which are identified due to the protein-protein interactions, methods for screening drugs for agents which modulate the interaction of proteins and pharmaceutical compositions that are capable of modulating the protein-protein interactions.

### CROSS REFERENCE TO RELATED APPLICATIONS

This application claims priority from U.S. provisional application No. 60/299,848 filed Jun. 21, 2001 and European application No. 02290277.9 filed Feb. 5, 2002.

Most biological processes involve specific protein-protein interactions. Protein-protein interactions enable two or more proteins to associate. A large number of non-covalent bonds form between the proteins when two protein surfaces are precisely matched. These bonds account for the specificity of recognition. Thus, protein-protein interactions are involved, for example, in the assembly of enzyme subunits, in antibody-antigen recognition, in the formation of biochemical complexes, in the correct folding of proteins, in the metabolism of proteins, in the transport of proteins, in the localization of proteins, in protein turnover, in first translation modifications, in the core structures of viruses and in signal transduction.

General methodologies to identify interacting proteins or to study these interactions have been developed. Among these methods are the two-hybrid system originally developed by Fields and co-workers and described, for example, in U.S. Pat. Nos. 5,283,173, 5,468,614 and 5,667,973, which are hereby incorporated by reference.

The earliest and simplest two-hybrid system, which acted as basis for development of other versions, is an in vivo assay between two specifically constructed proteins. The first protein, known in the art as the “bait protein” is a chimeric protein which binds to a site on DNA upstream of a reporter gene by means of a DNA-binding domain or BD. Commonly, the binding domain is the DNA-binding domain from either Gal4 or native E. coli LexA and the sites placed upstream of the reporter are Gal4 binding sites or LexA operators, respectively.

The second protein is also a chimeric protein known as the “prey” in the art. This second chimeric protein carries an activation domain or AD. This activation domain is typically derived from Gal4, from VP16 or from B42.

Besides the two-hybrid systems, other improved systems have been developed to detected protein-protein interactions. For example, a two-hybrid plus one system was developed that allows the use of two proteins as bait to screen available cDNA libraries to detect a third partner. This method permits the detection between proteins that are part of a larger protein complex such as the RNA polymerase II holoenzyme and the TFIIH or TFIID complexes. Therefore, this method, in general, permits the detection of ternary complex formation as well as inhibitors preventing the interaction between the two previously defined fused proteins.

Another advantage of the two-hybrid plus one system is that it allows or prevents the formation of the transcriptional activator since the third partner can be expressed from a conditional promoter such as the methionine-repressed Met25 promoter which is positively regulated in medium lacking methionine. The presence of the methionine-regulated promoter provides an excellent control to evaluate the activation or inhibition properties of the third partner due to its “on” and “off” switch for the formation of the transcriptional activator. The three-hybrid method is described, for example in Tirode et al., The Journal of Biological Chemistry, 272, No. 37 pp. 22995-22999 (1997) incorporated herein by reference.

Besides the two and two-hybrid plus one systems, yet another variant is that described in Vidal et al, Proc. Natl. Sci. 93 pgs. 10315-10320 called the reverse two- and one-hybrid systems where a collection of molecules can be screened that inhibit a specific protein-protein or protein-DNA interactions, respectively.

A summary of the available methodologies for detecting protein-protein interactions is described in Vidal and Legrain, Nucleic Acids Research Vol. 27, No. 4 pgs. 919-929 (1999) and Legrain and Selig, FEBS Letters 480 pgs. 32-36 (2000) which references are incorporated herein by reference.

However, the above conventionally used approaches and especially the commonly used two-hybrid methods have their drawbacks. For example, it is known in the art that, more often than not, false positives and false negatives exist in the screening method. In fact, a doctrine has been developed in this field for interpreting the results and in common practice an additional technique such as co-immunoprecipitation or gradient sedimentation of the putative interactors from the appropriate cell or tissue type are generally performed. The methods used for interpreting the results are described by Brent and Finley, Jr. in Ann. Rev. Genet., 31 pgs. 663-704 (1997). Thus, the data interpretation is very questionable using the conventional systems.

One method to overcome the difficulties encountered with the methods in the prior art is described in WO99/42612, incorporated herein by reference. This method is similar to the two-hybrid system described in the prior art in that it also uses bait and prey polypeptides. However, the difference with this method is that a step of mating at least one first haploid recombinant yeast cell containing the prey polypeptide to be assayed with a second haploid recombinant yeast cell containing the bait polynucleotide is performed. Of course the person skilled in the art would appreciate that either the first recombinant yeast cell or the second recombinant yeast cell also contains at least one detectable reporter gene that is activated by a polypeptide including a transcriptional activation domain.

The method described in WO99/42612 permits the screening of more prey polynucleotides with a given bait polynucleotide in a single step than in the prior art systems due to the cell to cell mating strategy between haploid yeast cells. Furthermore, this method is more thorough and reproducible, as well as sensitive. Thus, the presence of false negatives and/or false positives is extremely minimal as compared to the conventional prior art methods.

Deafness can be due to genetic or environmental causes or a combination of both. The main contributing environmental factors are meningitis, mumps, perinatal complications, maternofetal infection, acoustic trauma and ototoxic drug (Kalatzis and Petit, 1998, The fundamental and medical impacts of recent progress in research on hereditary hearing loss, Hum. Mol. Genet., 7, 1589-1597).

It has been estimated that 60% of the cases without an obvious environmental origin have a genetic basis. Syndromic hearing loss can have many modes of transmission, including maternal inheritance due to mitochondrial mutation.

Forms of deafness seem to be either autosomal dominant or maternally inherited due to mitochondrial mutations (Prezent et al., 1993, Mitochondrial ribosomal RNA mutation associated with both antibiotic-induced and non-syndromic deafness. Nature Genet., 4, 289-294, Reid et al., 1994, A novel mitochondrial point mutation in a maternal pedigree with sensorinal deafness, Hum. Mutat., 3, 243-247, Fischel-Ghodsian et al., 1995, Mitochondrial mutation associated with nonsyndromic deafness, Am. J. Otolaryngol., 16, 403-408). The autosomal recessive forms are rare. These forms also seem to be mainly sensorineural defects and are often progressive. Among the late onset forms, osteosclerosis is the most common cause of hearing impairment, this disorder has an autosomal dominant mode of transmission with incomplete penetrance.

To date, around 35 genetic loci have been mapped. As more than one locus has been assigned to the same chromosomal region, it is believed that the same locus may have been given two names. As the known loci do not account for all of the families studied to date, it seems that there still remain a significant number of unidentified loci underlying isolated forms of hearing loss.

It is admitted that in most instances, the identification of a deafness genes provides limited information. Specific studies are encountered in understanding the role of each of the proteins encoded by these genes.

Deaf-blindness in three forms of Usher type I syndrome (USH1) is caused by defects in myosin VIIa, the PDZ-protein harmonin, and cadherin 23. Despite being critical for hearing, the functions of these proteins in the ear remain largely elusive. In the present invention it is shown that all three are components of the mechanosensory hair bundle. Harmonin b is found to be an F-actin bundling protein that binds to the cytoplasmic domain of cadherin 23, thereby anchoring this adhesion molecule of the hair-bundles' surface to the actin-rich cores of its stereocilia. Moreover, harmonin b is absent from the disorganized hair bundles of myosin VIIa mutant mice, and interacts directly with myosin VIIa, suggesting myosin VIIa conveys harmonin b to the hair bundle. It can be concluded that an early inter-stereocilial adhesion process is required to shape a coherent hair bundle. It relies on myosin VIIa, harmonin b and cadherin 23 acting together as a functional network.

This “Usher” network also includes the protein vezatin that has recently been shown by to bind myosin VIIA (see, Kussel-Andermann et al., The Embo Journal Vol. 19, No. 22 pp. 6020-6029 (2000)). This novel membrane adhesion complex plays an important role in others cellular process that involve cell adhesion such as tumoral invasion and cellular invasion by bacteria. In particular, the cadherins have been involved in the regulation of cell proliferation, invasion, and intracellular signaling during cancer progression (Conacci-Sorrell M. et al. Journal of Clinical Investigation, 2002, 109 :987)

A better knowledge of proteins interactions in such pathways, as evidenced in the present invention, can help the development of new treatments. Available treatments are the amplification of sound or stimulation of the cochlear nerve or nucleus via cochlear or auditory brainstem implants respectively. But the development of new treatments, such as gene therapy, will only be possible when a minimal amount of knowledge concerning each of these defective processes will have been accumulated.

This shows that it is still needed to explore all mechanisms of inner ear cells and to identify drug targets for deafness and hearing disorders and/or diseases.

### SUMMARY OF THE INVENTION

Thus, an aspect of the present invention to identify protein-protein interactions of proteins expressed in inner ear cells involved in hearing disorders and/or diseases.

It is another aspect of the present invention to identify protein-protein interactions involved in hearing disorders and/or diseases for the development of more effective and better-targeted therapeutic treatments.

It is yet another aspect of the present invention to identify a functional network that is disrupted in Usher type 1 B syndrome, Usher type 1 C syndrome and Usher type 1 D syndrome.

It is yet another aspect of the present invention to identify complexes of polypeptides or polynucleotides encoding the polypeptides and fragments of the polypeptides of inner ear cells.

It is yet another aspect of the present invention to identify antibodies to these complexes of polypeptides or polynucleotides encoding the polypeptides and fragments of the polypeptides of the inner ear including polyclonal, as well as monoclonal antibodies that are used for detection.

It is still another aspect of the present invention to identify selected interacting domains of the polypeptides, called SID® polypeptides.

It is still another aspect of the present invention to identify selected interacting domains of the polynucleotides, called SID® polynucleotides.

It is another aspect of the present invention to generate protein-protein interactions maps called PIM®s.

It is yet another aspect of the present invention to provide a method for screening drugs for agents which modulate the interaction of proteins and pharmaceutical compositions that are capable of modulating the protein-protein interactions involved in hearing disorders and/or diseases.

It is another aspect to administer the nucleic acids of the present invention via gene therapy.

It is yet another aspect of the present invention to provide protein chips or protein microarrays.

It is yet another aspect of the present invention to provide a report in, for example paper, electronic and/or digital forms, concerning the protein-protein interactions, the modulating compounds and the like as well as a PIM®.

Thus the present invention relates to a complex of interacting proteins of columns 1 and 3 of Table 2.

Furthermore, the present invention provides SID® polynucleotides and SID® polypeptides, as well as a PIM® involved in hearing disorders and/or diseases.

The present invention also provides antibodies to the protein-protein complexes involved in hearing disorders and/or diseases.

The present invention also identifies the proteins in the inner ear that are required to shape a coherent hair bundle which are myosin VIIa, harmonin b and cadherin 23.

The present invention also identifies a functional network that is disrupted in Usher type 1 B syndrome, Usher type 1 C syndrome and Usher type 1 D syndrome which are myosin VIIa, harmonin b and cadherin 23.

In another embodiment the present invention provides a method for screening drugs for agents that modulate the protein-protein interactions and pharmaceutical compositions that are capable of modulating protein-protein interactions.

In another embodiment the present invention provides protein chips or protein microarrays.

In yet another embodiment the present invention provides a report in, for example, paper, electronic and/or digital forms.

### BRIEF DESCRIPTION OF THE DRAWINGS

The patent or application file contains at least one drawing executed in color. Copies of this patent or patent application publication with color drawings will be provided by the Patent and Trademark Office upon request and payment of necessary fee.

FIG. 1 is a schematic representation of the pB1 plasmid.

FIG. 2 is a schematic representation of the pB5 plasmid.

FIG. 3 is a schematic representation of the pB6 plasmid.

FIG. 4 is a schematic representation of the pB13 plasmid.

FIG. 5 is a schematic representation of the pB14 plasmid.

FIG. 6 is a schematic representation of the pB20 plasmid.

FIG. 7 is a schematic representation of the pP1 plasmid.

FIG. 8 is a schematic representation of the pP2 plasmid.

FIG. 9 is a schematic representation of the pP3 plasmid.

FIG. 10 is a schematic representation of the pP6 plasmid.

FIG. 11 is a schematic representation of the pP7 plasmid.

FIG. 12 is a schematic representation of vectors expressing the T25 fragment.

FIG. 13 is a schematic representation of vectors expressing the T18 fragment.

FIG. 14 is a schematic representation of various vectors of pCmAHL1, pT25 and pT18.

FIG. 15 is a schematic representation identifying the SID®'s of proteins of brain posterior bloc cells. In this figure the “Full-length prey protein” is the Open Reading Frame (ORF) or coding sequence (CDS) where the identified prey polypeptides are included. The Selected Interaction Domain (SID®) is determined by the commonly shared polypeptide domain of every selected prey fragment.

FIG. 16 is a protein map (PIM®).

FIG. 17 is a schematic representation of the pB27 plasmid.

FIG. 18(A) is a drawing illustrating Myosin VIIa that consists of a motor head that contains the ATP- and actin-binding sites, a neck region composed of 5 isoleucine-glutamine (IQ) motifs and a long tail. The tail begins with a dimerization domain (α-helix) followed by two MyTH4 (myosin tail homology)+FERM (4.1, ezrin, radixin, moesin)repeats, separated by an SH3 (src homology-3) domain.

Three classes of harmonin isoforms are reported: class a, which contains three PDZ (Post synaptic density, Disc large, Zonula occludens) domains and one coiled-coil (CC1) domain. Class b isoforms contain an additional coiled-coil domain (CC2) and a proline, serine, threonine(PST)-rich region. Class C isoforms contain only the first two PDZ domains and the first coiled-coil domain.

FIG. 18B is a schematic representation of a sensory hair cell, The height of sequential ranks of stereocilia increase from one side of the hair bundle to the other. The stereocilia insert into the dense network of actin filaments, the cuticular plate. At the apex of stereocilia, the tip links extends obliquely from the tip of a stereocilium, and is attached to the mechanotransduction channel of the tallest stereocilium, The actin skeleton is shown in red.

FIG. 18C is an immunofluorescence photograph showing myosin VIIa, cadherin 23 and harmonin in the inner ear. At P8, in the vestibular macula, myosin VIIa is observed throughout the cell body and spread along the entire length of stereocilia.

FIG. 18D is an immunofluorescence photograph showing myosin VIIa, cadherin 23 and harmonin in the inner ear and the location of harmonin b located at the tip of stereocilia.

FIG. 18E is an immunofluorescence photograph showing myosin VIIa, cadherin 23 and harmonin in the inner ear. At P8, in the vestibular macula, myosin VIIa is observed throughout the cell body and spread along the entire length of stereocilia.

FIG. 18F is an immunofluorescence photograph showing myosin VIIa, cadherin 23 and harmonin in the inner ear and the location of harmonin b located at the tip of stereocilia.

FIG. 18G is an immunofluorescence photograph showing myosin VIIa, cadherin 23 and harmonin in the inner ear and t later stages, harmonin b persists at the tip of some hair bundles at P30.

FIG. 18H is an immunofluorescence photograph that demonstrates that cadherin 23 is also present in hair bundles.

FIG. 18I is an immunofluorescence photograph that demonstrates that cadherin 23 is enriched in the tips of the stereocilia that are visualized by F-actin staining at P8.

FIG. 18J is an immunofluorescence photograph that demonstrates that cadherin 23 is enriched in the tips of the stereocilia that are visualized by F-actin staining at P8.

FIG. 18K is an immunofluorescence photograph that demonstrates that cadherin 23 is enriched in the tips of the stereocilia that are visualized by F-actin staining at P30.

FIG. 18L is an immunofluorescence photograph that demonstrates that cadherin 23 is also present in hair bundles.

FIG. 18M is an immunofluorescence photograph that demonstrates that cadherin 23 is enriched in the tips of the stereocilia that are visualized by F-actin staining at P30.

FIG. 18N is an immunofluorescence photograph that demonstrates that cadherin 23 is enriched in the tips of the stereocilia that are visualized by F-actin staining at P30. Bars are 10 μm in these figures.

FIG. 19(A) is a schematic representation of the auditory sensory organ, organ of Corti. It is made up of sensory cells, the inner (ihc) and the outer (ohc) hair cells (in red), flanked by various epithelial supporting cells (in blue).

FIG. 19B is an immunofluorescence photograph showing a rat organ of Corti at P4 showing that Myosin VIIa is present in the sensory hair cells, both in the cell body and in the hair bundle.

FIG. 19C is an immunofluorescence photograph showing a rat organ of Corti at P4. Using the NW1 antibody, harmonin isoforms can be detected in the sensory hair cells (asterisks over hair cell bodies), with the labeling being present in the cuticular plate as well as in the apical hair bundle.

FIG. 19D is an immunofluorescence photograph showing a rat organ of Corti at P4 showing harmonin b isoform is present in the hair bundle only. Bars are 10 μm.

FIG. 20A is a electron microscope photograph showing vestibular sensory epithelia at E 14.

FIG. 20B is an immunofluorescence photograph showing vestibular sensory epithelia at E 14, whereas myosin VIIa is present throughout the hair cell.

FIG. C is an immunofluorescence photograph showing vestibular sensory epithelia at E 14 and harmonin b detected at the apical hair cell surface only.

FIG. 20D is an immunofluorescence photograph showing vestibular sensory epithelia at E 14 and harmonin b detected at the apical hair cell surface only.

FIG. 20E is an electron microscope photograph showing vestibular sensory epithelia at E 14 and that harmonin b labeling is also present around the cuticular plate.

FIG. 20F is an immunofluorescence photograph showing vestibular sensory epithelia at E 14, whereas myosin VIIa is present throughout the hair cell.

FIG. 20G is an immunofluorescence photograph showing vestibular sensory epithelia at E 14 and cadherin 23 detected at the apical hair cell surface only.

FIG. 20H is an immunofluorescence photograph showing vestibular sensory epithelia at E 14 and cadherin 23 detected at the apical hair cell surface only.

FIG. 20I is an immunofluorescence photograph showing that at E 16 in the crista ampullaris, sensory hair cells express harmonin b along the entire length of the hair bundles visualized by F-actin staining.

FIG. 20J is an immunofluorescence photograph showing that at E 16 in the crista ampullaris, sensory hair cells express cadherin 23 along the entire length of the hair bundles visualized by F-actin staining.

FIG. 20K is an immunofluorescence photograph showing that at E 16 in the crista ampullaris, sensory hair cells express cadherin 23 along the entire length of the hair bundles visualized by F-actin staining.

FIG. 21 is an immunofluorescence photograph that demonstrates that at P0, in the cochlea, either harmonin b (A) or cadherin 23 (B) are enriched at the tips of the stereocilia (arrowheads).

Bars are 10 μm.

FIG. 22A is a schematic picture showing details of the hair bundle. Two stereocilia are represented. Each stereocilium is filled with several actin filaments that are cross-linked by fimbrin and espin. Only the central actin filaments of the stereocilia extend rootlets into the cuticular plate. Four different types of lateral; links interconnect the stereocilia: tip links (TL), horizontal top links (HL), shaft links (SSL) and ankle links (AL).

FIG. 22B is an immunofluorescence photograph showing the ultrastructural distribution of cadherin 23 in the hair bundle. Using the cad-N antibody, cadherin 23 is detected between adjacent stereocilia.

FIG. 22C is an immunofluorescence photograph showing the ultrastructural distribution of cadherin 23 in the hair bundle. Using the cad-N antibody, cadherin 23 is detected between adjacent stereocilia, as shown in the higher magnification (arrows),

FIG. 22D is an immunofluorescence photograph showing whole-mount preparation of a mouse organ of Corti labeled for cadherin 23. In control cultures, the cad-N anti-cadherin 23 antibody labels the hair bundle of the inner (U-shape) and outer (V-shape hair cells.

FIG. 22E is an immunofluorescence photograph showing that upon BAPTA treatment the cadherin 23 labeling is unaffected.

FIG. 22F is an immunofluorescence photograph showing that no labeling is observed when the organ of Corti is treated with subtilisin.

FIG. 23A is an immunofluorescence photograph showing that when transiently transfected HeLa cells, harmonin a is distributed throughout the cytosol.

FIG. 23B is an immunofluorescence photograph showing that when transiently transfected HeLa cells, harmonin b is distributed throughout the cytosol and that over expression of harmonin b leads to the formation of curvy bundles.

FIG. 23C is an immunofluorescence photograph showing that when transiently transfected HeLa cells, harmonin b is distributed throughout the cytosol and that over expression of harmonin b leads to the formation of curvy bundles, which colocalizes with TRITC-phalloiden labeling.

FIG. 23D is an immunofluorescence photograph showing that when transiently transfected HeLa cells, harmonin b is distributed throughout the cytosol and that over expression of harmonin b leads to the formation of curvy bundles, which colocalizes with TRITC-phalloiden labeling.

FIG. 23E is an immunofluorescence photograph showing that at low expression levels, a triple staining with TRITC-phalloidine, anti-vinculin and anti-harmonin b antibodies reveals that harmonin b decorates the extremity of actin stress fibers.

FIG. 23F is an immunofluorescence photograph showing that at low expression levels, a triple staining with TRITC-phalloidine, anti-vinculin and anti-harmonin b antibodies reveals that harmonin b decorates the extremity of actin stress fibers and are anchored to focal adhesion plaques.

FIG. 23G is an immunofluorescence photograph showing that at low expression levels, a triple staining with TRITC-phalloidine, anti-vinculin and anti-harmonin b antibodies reveals that harmonin b decorates the extremity of actin stress fibers and are anchored to focal adhesion plaques, but does not colocalize with vinculin staining.

FIG. 23H is an immunofluorescence photograph showing that at low expression levels, a triple staining with TRITC-phalloidine, anti-vinculin and anti-harmonin b antibodies reveals that harmonin b decorates the extremity of actin stress fibers and are anchored to focal adhesion plaques, but does not colocalize with vinculin staining. Bars are 20μm in FIG. 23.

FIG. 24 A is an immunofluorescence photograph showing a control of HeLa cells labeled for harmonin b and F-actin.

FIG. 24 B is an immunofluorescence photograph showing that actin stress fibers are observed in either harmonin b transfected cells or in the surrounding untransfected cells, after cytochalasin D (CyD) treatment, bound F-actin filaments are only observed in cells transfected with harmonin b.

FIG. 24C is an immunofluorescence photograph showing that actin stress fibers are observed in either harmonin b transfected cells or in the surrounding untransfected cells, after cytochalasin D (CyD) treatment, bound F-actin filaments are only observed in cells transfected with harmonin b.

FIG. 24D is an immunofluorescence photograph showing that actin stress fibers are observed in either harmonin b transfected cells or in the surrounding untransfected cells, after cytochalasin D (CyD) treatment, bound F-actin filaments are only observed in cells transfected with harmonin b. Cosedimentation of His-tagged harmonin b with F-actin.

FIG. 24E shows a light microscopy of F-actin filament collected in the presence of harmonin b.

FIG. 24E′ shows a light microscopy of F-actin filament collected in the absence of harmonin b.

FIG. 24F shows an electron microscopy in which large F-actin bundles are obtained in the presence of harmonin b, thus showing the F-actin bundling activity of this protein.

FIG. 24G shows an electron microscopy in which large F-actin bundles are obtained in the presence of harmonin b, thus showing the F-actin bundling activity of this protein. For FIG. 24 bars are 10 μm for FIGS. A to C and 50 nm for FIGS. F and G.

FIG. 25A is a gel showing the results of a pull down assay. The full lengths harmonin a and b bind to the GST-tagged cadherin 23 cytodomain but not to GST alone.

FIG. 25B is a gel showing that the myosin VIIa tail does not bind to cadherin 23in in vitro binding assays. The dissection of the harmonin-cadherin 23 interaction is also shown. The PDZ2 domain of harmonin is sufficient to bind to an immobilized biotin-tagged cadherin 23 cytodomain. Biotin-tagged CAT (chloramphenical Acetyl Transferase) is used as a negative control.

FIG. 25C is a gel showing that myosin VIIa-Cter binds to GST-harmonin a, but not to GST alone. The FERM domain of ezrin is used as a negative control and MyRIP and a myosin VIIa interacting protein, as a positive control.

FIG. 25D is a gel showing a dissection of the harmonin-myosin VIIa interaction which indicates that the PDZ1 domain of harmonin binds to the cadherin 23 tail but not PDZ2 nor PDZ3.

FIG. 26 A is an immunofluorescence photograph showing harmonin b colocalized with cadherin 23, myosin VIIa and actin in HeLa transfected cells. The presence of the cadherin 23 cytodomain in cotransfected cells shifts the filamentous pattern usually observed with harmonin b, to punctiform actin rich structures.F-actin is visualized with TRITC-phalloidin staining, harmonin b with the H1b antibody, cadherin cytodomain with myc antibody; and myosin VIIa with the monoclonal mouse antibody.

FIG. 26B is a schematic representation of yeast two-hybrid harmonin preys isolated using the cadherin 23 cytodomain (aa 3086-3354) as the bait.

FIG. 26C is an immunofluorescence photograph showing harmonin b colocalized perfectly with the myosin VIIa tail-actin filament structures.

FIG. 26D is a schematic representation of Yeast two-hybrid harmonin preys obtained using the SH3-MyTH4-FERM domains of myosin VIIa (aa 1562-2215) as the bait.

The gray box indicate the selected interacting domain based on the prey clones: 16 for cadherin 23, and 6 for myosin VIIa. Bars are 20 μm for all of FIGS. 26.

FIG. 27 A is an immunofluorescence photograph showing vestibular hair cells from shaker-1 Myo7a^{4626SB} mutant mice at P8. Harmonin b decorates the apical surface of the sensory hair cell around the cuticular plate. No harmonin b labeling is observed in the hair bundles that are visualized by the F-actin staining.

FIG. 27B is an immunofluorescence photograph showing vestibular hair cells from shaker-1 Myo7a^{4626SB} mutant mice at P8. Harmonin b decorates the apical surface of the sensory hair cell around the cuticular plate. No harmonin b labeling is observed in the hair bundles that are visualized by the F-actin staining.

FIG. 27C is an immunofluorescence photograph showing vestibular hair cells from shaker-1 Myo7a^{4626SB} mutant mice at P8. Harmonin b decorates the apical surface of the sensory hair cell around the cuticular plate. No harmonin b labeling is observed in the hair bundles that are visualized by the F-actin staining.

FIG. 27D is an immunofluorescence photograph showing vestibular hair cells from shaker-1 Myo7a^{4626SB} mutant mice at P8. Harmonin b decorates the apical surface of the sensory hair cell around the cuticular plate. No harmonin b labeling is observed in the hair bundles that are visualized by the F-actin staining.

FIG. 27E is an immunofluorescence photograph showing vestibular hair cells from shaker-1 Myo7a^{4626SB} mutant mice at P8. Harmonin b decorates the apical surface of the sensory hair cell around the cuticular plate. No harmonin b labeling is observed in the hair bundles that are visualized by the F-actin staining.

FIG. 27F is an immunofluorescence photograph showing that although vestibular hair cells from shaker-1 Myo7a^{4626SB} mutant mice at P8. Harmonin b decorates the apical surface of the sensory hair cell around the cuticular plate. No harmonin b labeling is observed in the hair bundles that are visualized by the F-actin staining, stereocilin is detected along the entire length of the stereocilia.

FIG. 27G is an immunofluorescence photograph showing cochlear hair cells from shaker-1 Myo7a^{4626SB} mutant mice at P6. In the cochlea, harmonin b fails to attain the hair bundle and punctuated harmonin b labeled structures are observed around and within the cuticular plate.

FIG. 27H is an immunofluorescence photograph showing cochlear hair cells from shaker-1 Myo7a^{4626SB} mutant mice at P6. In the cochlea, harmonin b fails to attain the hair bundle and punctuated harmonin b labeled structures are observed around and within the cuticular plate.

FIG. 27I is an immunofluorescence photograph showing that in an adjacent section, all three harmonin isoforms are labeled using the NW2 antibody.

FIG. 27J is an immunofluorescence photograph showing a higher magnification of views of vestibular hair bundles from P2 mice. In control mice, harmonin b is essentially located at the apex of the myosin VIIa- or F-actin labeled hair bundles.

FIG. 27K is an immunofluorescence photograph showing in Myo7a^{4626SB} mutant mice, harmonin b is mainly organized in a circle of bead-like foci located between the actin-rich cuticular plate and the actin of the circumferential belt.

FIG. 27L is an immunofluorescence photograph showing a higher magnification of views of vestibular hair bundles from P2 mice. In these mice, espin is properly targeted to the hair bundle where it is expected to cross link the actin filament (F-actin) of stereocilia.

FIG. 27M is an immunofluorescence photograph showing a higher magnification of views of cochlear hair bundles from P2 mice. In control mice, harmonin b is essentially located at the apex of the myosin VIIa- or F-actin labeled hair bundles.

FIG. 27N is an immunofluorescence photograph showing a higher magnification of views of cochlear hair bundles from P2 mice. In Myo7a^{4626SB} mutant mice, harmonin b is mainly organized in a circle of bead-like foci located between the actin-rich cuticular plate and the actin of the circumferential belt.

FIG. 27O is an immunofluorescence photograph showing a higher magnification of views of cochlear hair bundles from P2 mice. In these mice, espin is properly targeted to the hair bundle where it is expected to cross link the actin filament (F-actin) of stereocilia.

FIG. 28A is a schematic picture showing details of the hair bundle showing the four different types of stereocilia lateral links described in chick: tip links (TL), horizontal top links (HL), shaft links, (SL) and the ankle links (AL).

FIG. 28B is an electron microscopy of stereocilia from control mouse organ of Corti cultures.

FIG. 28C is an electron microscopy of stereocilia from control mouse organ of Corti cultures in the presence of BAPTA.). Unlike the cadherin 23 links, the horizontal top links are BAPTA/subtilisin insensitive.

FIG. 28D is an electron microscopy of stereocilia from control mouse organ of Corti cultures in the presence of subtilisin.). Unlike the cadherin 23 links, the horizontal top links are BAPTA/subtilisin insensitive.

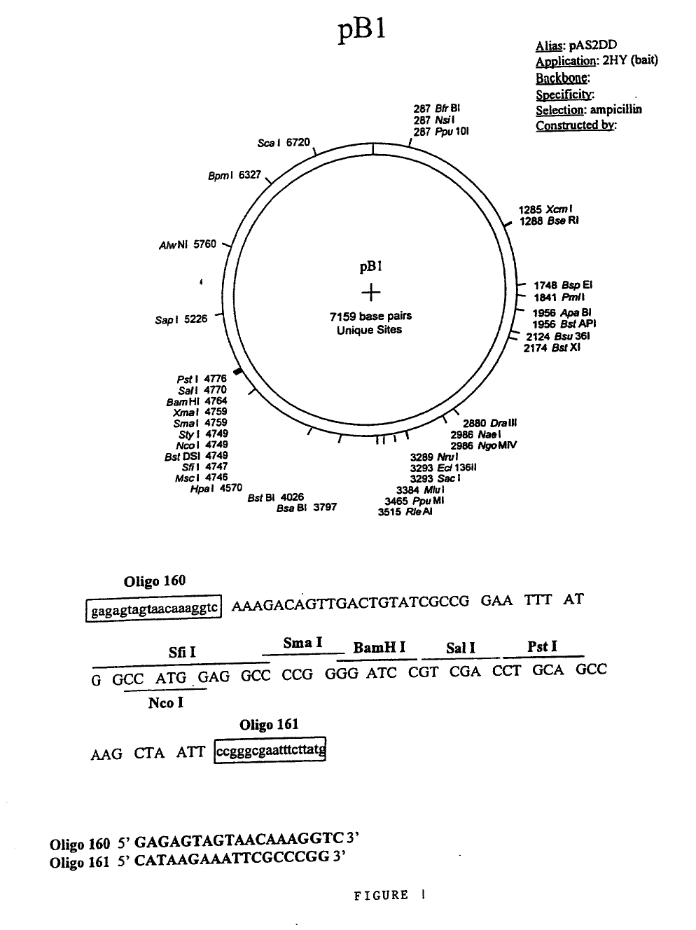

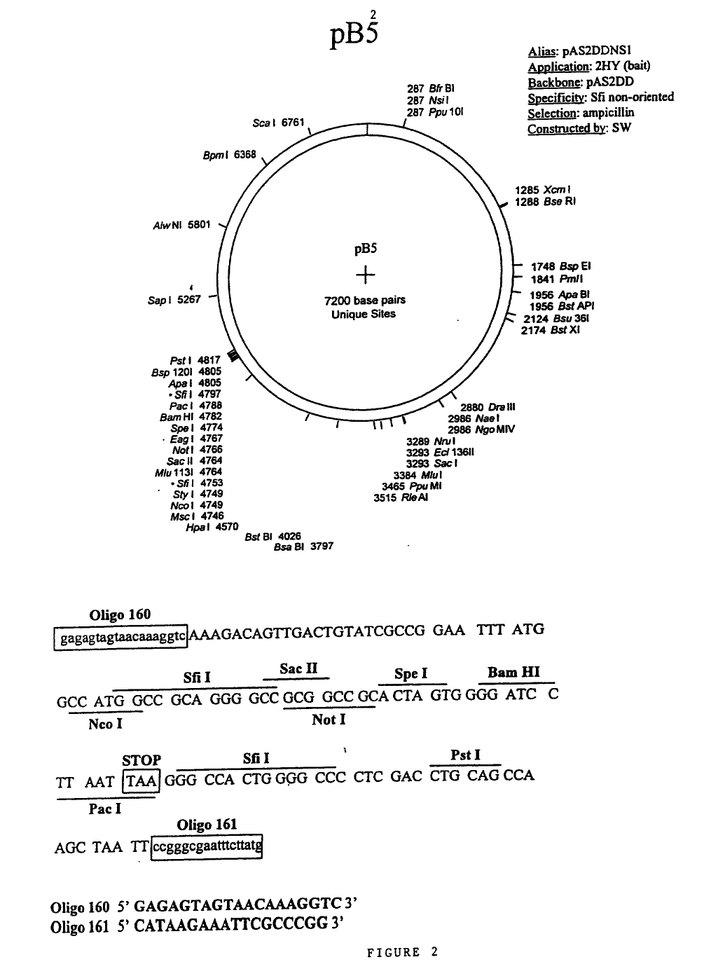

### DETAILED DESCRIPTION

As used herein the terms “polynucleotides”, “nucleic acids” and “oligonucleotides” are used interchangeably and include, but are not limited to RNA, DNA, RNA/DNA sequences of more than one nucleotide in either single chain or duplex form. The polynucleotide sequences of the present invention may be prepared from any known method including, but not limited to, any synthetic method, any recombinant method, any ex vivo generation method and the like, as well as combinations thereof.

The term “polypeptide” means herein a polymer of amino acids having no specific length. Thus, peptides, oligopeptides and proteins are included in the definition of “polypeptide” and these terms are used interchangeably throughout the specification, as well as in the claims. The term “polypeptide” does not exclude post-translational modifications such as polypeptides having covalent attachment of glycosyl groups, acetyl groups, phosphate groups, lipid groups and the like. Also encompassed by this definition of “polypeptide” are homologs thereof.

By the term “homologs” is meant structurally similar genes contained within a given species, orthologs are functionally equivalent genes from a given species or strain, as determined for example, in a standard complementation assay. Thus, a polypeptide of interest can be used not only as a model for identifying similar genes in given strains, but also to identify homologs and orthologs of the polypeptide of interest in other species. The orthologs, for example, can also be identified in a conventional complementation assay. In addition or alternatively, such orthologs can be expected to exist in bacteria (or other kind of cells) in the same branch of the phylogenic tree, as set forth, for example, at ftp://ftp.cme.msu.edu/pub/rdp/SSU-rRNA/SSU/Prok.phylo.

As used herein the term “prey polynucleotide” means a chimeric polynucleotide encoding a polypeptide comprising (i) a specific domain; and (ii) a polypeptide that is to be tested for interaction with a bait polypeptide. The specific domain is preferably a transcriptional activating domain.

As used herein, a “bait polynucleotide” is a chimeric polynucleotide encoding a chimeric polypeptide comprising (i) a complementary domain; and (ii) a polypeptide that is to be tested for interaction with at least one prey polypeptide. The complementary domain is preferably a DNA-binding domain that recognizes a binding site that is further detected and is contained in the host organism.

As used herein “complementary domain” is meant a functional constitution of the activity when bait and prey are interacting; for example, enzymatic activity.

As used herein “specific domain” is meant a functional interacting activation domain that may work through different mechanisms by interacting directly or indirectly through intermediary proteins with RNA polymerase II or III-associated proteins in the vicinity of the transcription start site.

As used herein the term “complementary” means that, for example, each base of a first polynucleotide is paired with the complementary base of a second polynucleotide whose orientation is reversed. The complementary bases are A and T (or A and U) or C and G.

The term “sequence identity” refers to the identity between two peptides or between two nucleic acids. Identity between sequences can be determined by comparing a position in each of the sequences which may be aligned for the purposes of comparison. When a position in the compared sequences is occupied by the same base or amino acid, then the sequences are identical at that position. A degree of sequence identity between nucleic acid sequences is a function of the number of identical nucleotides at positions shared by these sequences. A degree of identity between amino acid sequences is a function of the number of identical amino acid sequences that are shared between these sequences. Since two polypeptides may each (i) comprise a sequence (i.e., a portion of a complete polynucleotide sequence) that is similar between two polynucleotides, and (ii) may further comprise a sequence that is divergent between two polynucleotides, sequence identity comparisons between two or more polynucleotides over a “comparison window” refers to the conceptual segment of at least 20 contiguous nucleotide positions wherein a polynucleotide sequence may be compared to a reference nucleotide sequence of at least 20 contiguous nucleotides and wherein the portion of the polynucleotide sequence in the comparison window may comprise additions or deletions (i.e., gaps) of 20 percent or less compared to the reference sequence (which does not comprise additions or deletions) for optimal alignment of the two sequences.

To determine the percent identity of two amino acids sequences or two nucleic acid sequences, the sequences are aligned for optimal comparison. For example, gaps can be introduced in the sequence of a first amino acid sequence or a first nucleic acid sequence for optimal alignment with the second amino acid sequence or second nucleic acid sequence. The amino acid residues or nucleotides at corresponding amino acid positions or nucleotide positions are then compared. When a position in the first sequence is occupied by the same amino acid residue or nucleotide as the corresponding position in the second sequence, the molecules are identical at that position.

The percent identity between the two sequences is a function of the number of identical positions shared by the sequences. Hence % identity=number of identical positions/total number of overlapping positions×100.

In this comparison the sequences can be the same length or may be different in length. Optimal alignment of sequences for determining a comparison window may be conducted by the local homology algorithm of Smith and Waterman (J. Theor. Biol., 91 (2) pgs. 370-380 (1981), by the homology alignment algorithm of Needleman and Wunsch, J. Miol. Biol., 48(3) pgs. 443-453 (1972), by the search for similarity via the method of Pearson and Lipman, PNAS, USA, 85(5) pgs. 2444-2448 (1988), by computerized implementations of these algorithms (GAP, BESTFIT, FASTA and TFASTA in the Wisconsin Genetics Software Package Release 7.0, Genetic Computer Group, 575, Science Drive, Madison, Wis.) or by inspection.

The best alignment (i.e., resulting in the highest percentage of identity over the comparison window) generated by the various methods is selected.

The term “sequence identity” means that two polynucleotide sequences are identical (i.e., on a nucleotide by nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size) and multiplying the result by 100 to yield the percentage of sequence identity. The same process can be applied to polypeptide sequences.

The percentage of sequence identity of a nucleic acid sequence or an amino acid sequence can also be calculated using BLAST software (Version 2.06 of September 1998) with the default or user defined parameter.

The term “sequence similarity” means that amino acids can be modified while retaining the same function. It is known that amino acids are classified according to the nature of their side groups and some amino acids such as the basic amino acids can be interchanged for one another while their basic function is maintained.

The term “isolated” as used herein means that a biological material such as a nucleic acid or protein has been removed from its original environment in which it is naturally present. For example, a polynucleotide present in a plant, mammal or animal is present in its natural state and is not considered to be isolated. The same polynucleotide separated from the adjacent nucleic acid sequences in which it is naturally inserted in the genome of the plant or animal is considered as being “isolated.”

The term “isolated” is not meant to exclude artificial or synthetic mixtures with other compounds, or the presence of impurities which do not interfere with the biological activity and which may be present, for example, due to incomplete purification, addition of stabilizers or mixtures with pharmaceutically acceptable excipients and the like.

“Isolated polypeptide” or “isolated protein” as used herein means a polypeptide or protein which is substantially free of those compounds that are normally associated with the polypeptide or protein in a naturally state such as other proteins or polypeptides, nucleic acids, carbohydrates, lipids and the like.

The term “purified” as used herein means at least one order of magnitude of purification is achieved, preferably two or three orders of magnitude, most preferably four or five orders of magnitude of purification of the starting material or of the natural material. Thus, the term “purified” as utilized herein does not mean that the material is 100% purified and thus excludes any other material.

The term “variants” when referring to, for example, polynucleotides encoding a polypeptide variant of a given reference polypeptide are polynucleotides that differ from the reference polypeptide but generally maintain their functional characteristics of the reference polypeptide. A variant of a polynucleotide may be a naturally occurring allelic variant or it may be a variant that is known naturally not to occur. Such non-naturally occurring variants of the reference polynucleotide can be made by, for example, mutagenesis techniques, including those mutagenesis techniques that are applied to polynucleotides, cells or organisms.

Generally, differences are limited so that the nucleotide sequences of the reference and variant are closely similar overall and, in many regions identical.

Variants of polynucleotides according to the present invention include, but are not limited to, nucleotide sequences which are at least 95% identical after alignment to the reference polynucleotide encoding the reference polypeptide. These variants can also have about 96%, 97%, 98% and 99.999% sequence identity to the reference polynucleotide.

Nucleotide changes present in a variant polynucleotide may be silent, which means that these changes do not alter the amino acid sequences encoded by the reference polynucleotide.

Substitutions, additions and/or deletions can involve one or more nucleic acids. Alterations can produce conservative or non-conservative amino acid substitutions, deletions and/or additions.

Variants of a prey or a SID® polypeptide encoded by a variant polynucleotide can possess a higher affinity of binding and/or a higher specificity of binding to its protein or polypeptide counterpart, against which it has been initially selected. In another context, variants can also loose their ability to bind to their protein or polypeptide counterpart.

By “anabolic pathway” is meant a reaction or series of reactions in a metabolic pathway that synthesize complex molecules from simpler ones, usually requiring the input of energy. An anabolic pathway is the opposite of a catabolic pathway.

As used herein, a “catabolic pathway” is a series of reactions in a metabolic pathway that break down complex compounds into simpler ones, usually releasing energy in the process. A catabolic pathway is the opposite of an anabolic pathway.

As used herein, “drug metabolism” is meant the study of how drugs are processed and broken down by the body. Drug metabolism can involve the study of enzymes that break down drugs, the study of how different drugs interact within the body and how diet and other ingested compounds affect the way the body processes drugs.

As used herein, “metabolism” means the sum of all of the enzyme-catalyzed reactions in living cells that transform organic molecules.

By “secondary metabolism” is meant pathways producing specialized metabolic products that are not found in every cell.

As used herein, “SID” means a Selected Interacting Domain and is identified as follows: for each bait polypeptide screened, selected prey polypeptides are compared. overlapping fragments in the same ORF or CDS define the selected interacting domain.

As used herein the term “PIM®” means a protein-protein interaction map. This map is obtained from data acquired from a number of separate screens using different bait polypeptides and is designed to map out all of the interactions between the polypeptides.

The term “affinity of binding”, as used herein, can be defined as the affinity constant Ka when a given SID® polypeptide of the present invention which binds to a polypeptide and is the following mathematical relationship:

[SID®/polypeptide complex]

Ka=---

[free SID®] [free polypeptide]

wherein [free SID®], [free polypeptide] and [SID®/polypeptide complex] consist of the concentrations at equilibrium respectively of the free SID® polypeptide, of the free polypeptide onto which the SID® polypeptide binds and of the complex formed between SID® polypeptide and the polypeptide onto which said SID® polypeptide specifically binds.

The affinity of a SID® polypeptide of the present invention or a variant thereof for its polypeptide counterpart can be assessed, for example, on a Biacore™ apparatus marketed by Amersham Pharmacia Biotech Company such as described by Szabo et al. (Curr Opin Struct Biol 5 pgs. 699-705 (1995)) and by Edwards and Leartherbarrow (Anal. Biochem 246 pgs. 1-6 (1997)).

As used herein the phrase “at least the same affinity” with respect to the binding affinity between a SID® polypeptide of the present invention to another polypeptide means that the Ka is identical or can be at least two-fold, at least three-fold or at least five fold greater than the Ka value of reference.

As used herein, the term “modulating compound” means a compound that inhibits or stimulates or can act on another protein which can inhibit or stimulate the protein-protein interaction of a complex of two polypeptides or the protein-protein interaction of two polypeptides.

More specifically, the present invention comprises complexes of polypeptides or polynucleotides encoding the polypeptides composed of a bait polypeptide, or a bait polynucleotide encoding a bait polypeptide and a prey polypeptide or a prey polynucleotide encoding a prey polypeptide. The prey polypeptide or prey polynucleotide encoding the prey polypeptide is capable of interacting with a bait polypeptide of interest in various hybrid systems.

As described in the Background of the present invention, there are various methods known in the art to identify prey polypeptides that interact with bait polypeptides of interest. These methods include, but are not limited to, generic two-hybrid systems as described by Fields et al. (Nature, 340:245-246 (1989)) and more specifically in U.S. Pat. Nos. 5,283,173, 5,468,614 and 5,667,973, which are hereby incorporated by reference; the reverse two-hybrid system described by Vidal et al. (supra); the two plus one hybrid method described, for example, in Tirode et al. (supra); the yeast forward and reverse ‘n’-hybrid systems as described in Vidal and Legrain (supra); the method described in WO 99/42612; those methods described in Legrain et al. (FEBS Letters 480 pgs. 32-36 (2000)) and the like.

The present invention is not limited to the type of method utilized to detect protein-protein interactions and therefore any method known in the art and variants thereof can be used. It is however better to use the method described in WO99/42612 or WO/0066722, both references incorporated herein by reference due to the methods' sensitivity, reproducibility and reliability.

Protein-protein interactions can also be detected using complementation assays such as those described by Pelletier et al. at http://www.abrf.org/JBT/Articles/JBT0012/jbt0012.html, WO 00/07038 and WO98/34120.

Although the above methods are described for applications in the yeast system, the present invention is not limited to detecting protein-protein interactions using yeast, but also includes similar methods that can be used in detecting protein-protein interactions in, for example, mammalian systems as described, for example in Takacs et al. (Proc. Natl. Acad. Sci., USA, 90 (21):10375-79 (1993)) and Vasavada et al. (Proc. Natl. Acad. Sci., USA, 88 (23 ):10686-90 (1991)), as well as a bacterial two-hybrid system as described in Karimova et al. (1998), WO99/28746, WO/0066722 and Legrain et al. (FEBS Letters, 480 pgs. 32-36 (2000)).

The above-described methods are limited to the use of yeast, mammalian cells and Escherichia coli cells, the present invention is not limited in this manner. Consequently, mammalian and typically human cells, as well as bacterial, yeast, fungus, insect, nematode and plant cells are encompassed by the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5-α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

The bait polynucleotide, as well as the prey polynucleotide can be prepared according to the methods known in the art such as those described above in the publications and patents reciting the known method per se.

The bait and the prey polynucleotide of the present invention is obtained from mouse's inner ear cells cDNA, or variants of cDNA fragment from a library of mouse's inner ear cells, and fragments from the genome or transcriptome of mousers inner ear cells cDNA ranging from about 12 to about 5,000, or about 12 to about 10,000 or from about 12 to about 20,000. The prey polynucleotide is then selected, sequenced and identified.

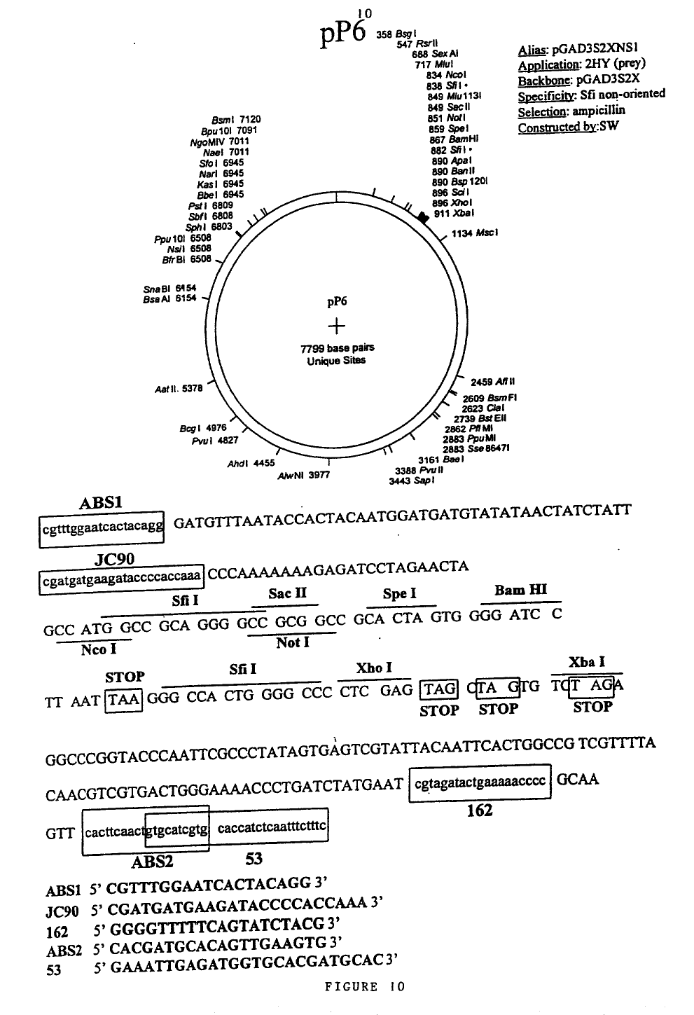

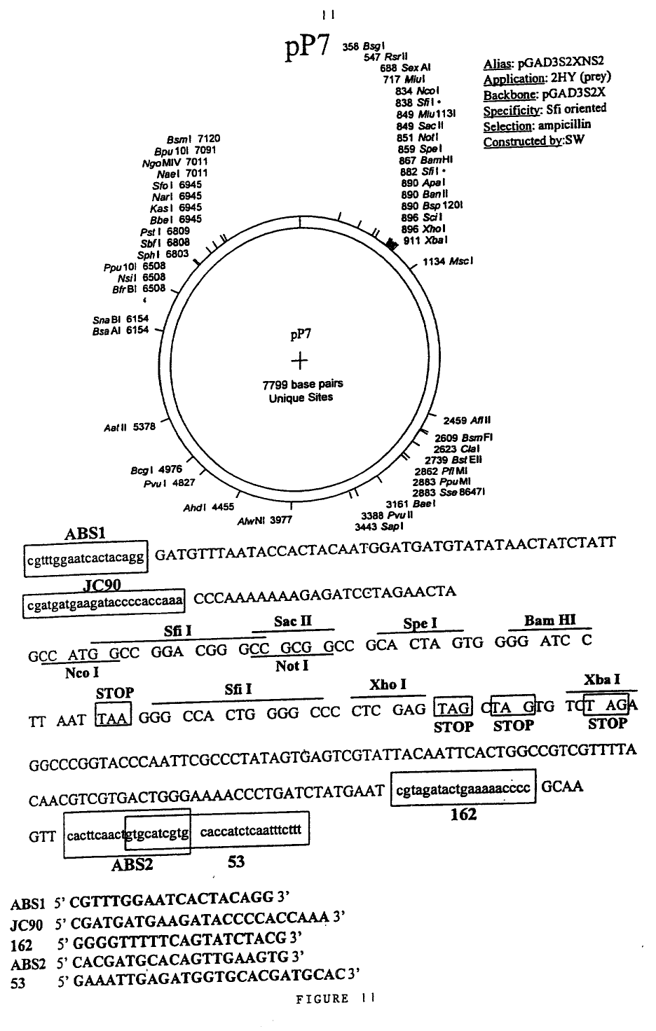

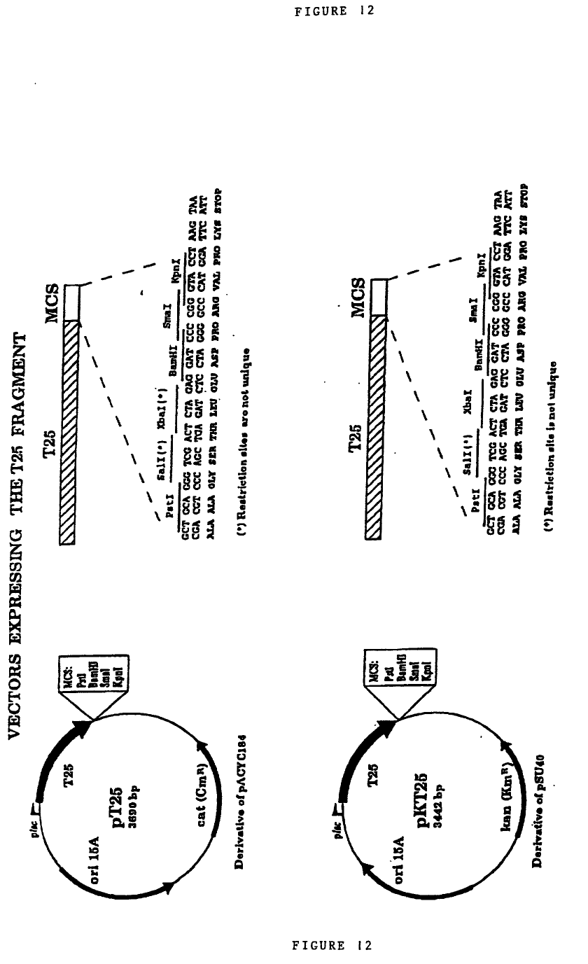

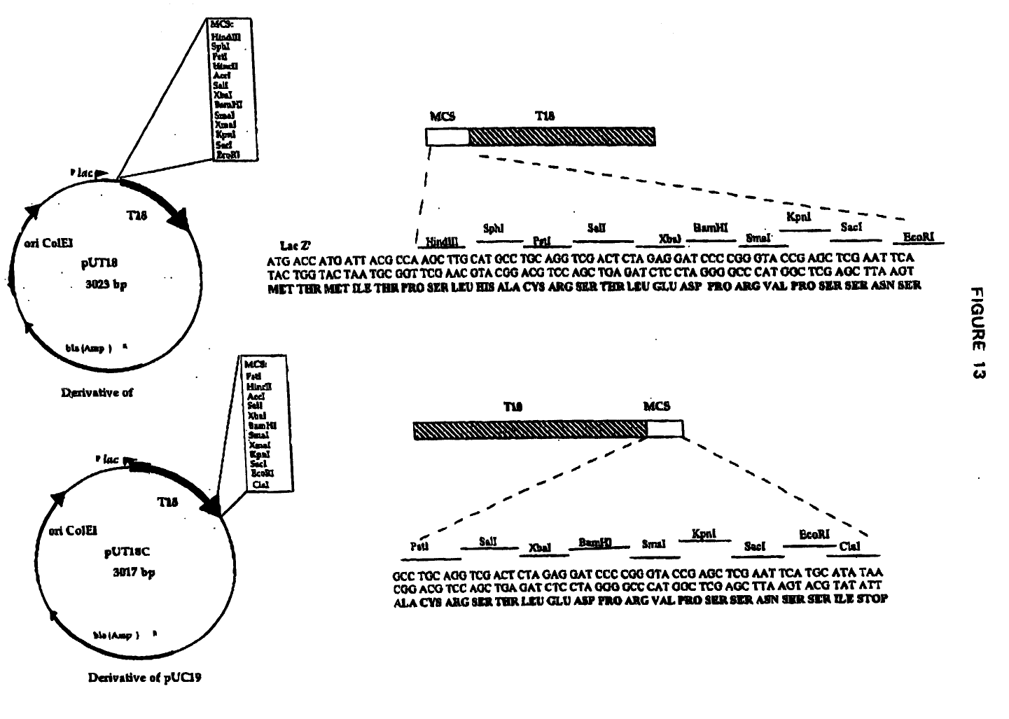

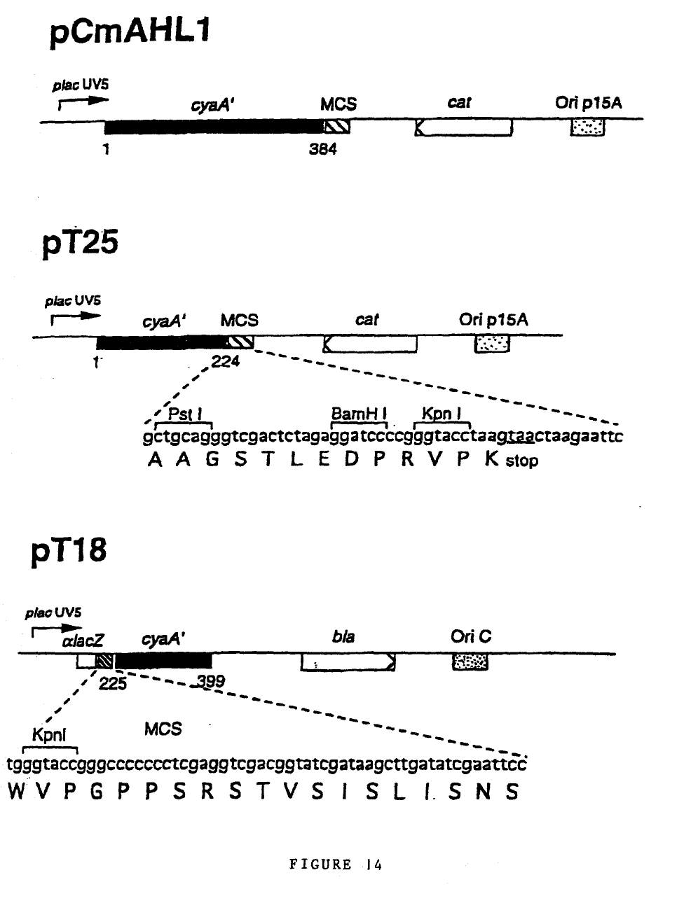

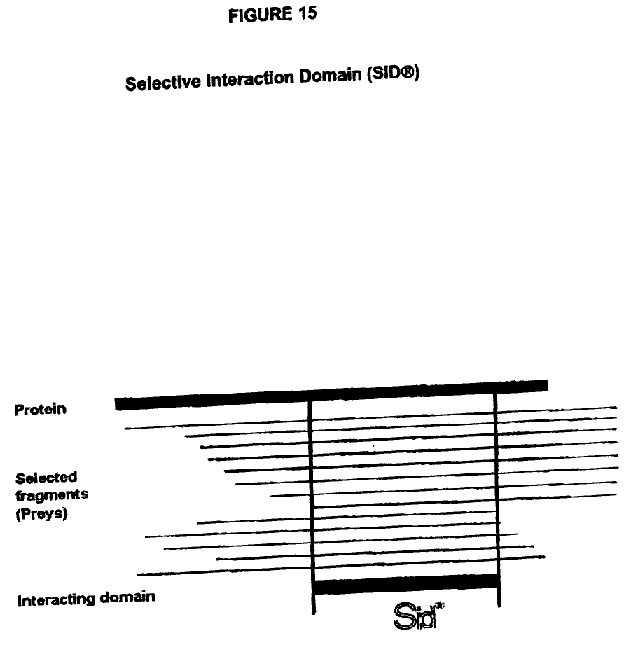

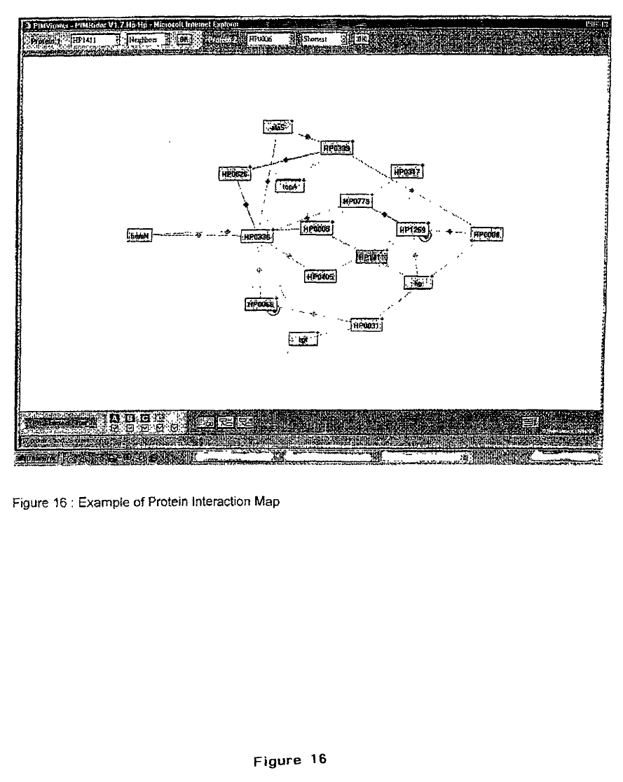

A rat's brain posterior bloc cells prey library is prepared from the rat's brain posterior bloc cells' cDNA and constructed in the specially designed prey vector pP6 as shown in FIG. 10 after ligation of suitable linkers such that every cDNA insert is fused to a nucleotide sequence in the vector that encodes the transcription activation domain of a reporter gene. Any transcription activation domain can be used in the present invention. Examples include, but are not limited to, Gal4,YP16, B42, His and the like. Toxic reporter genes, such as CAT^{R}, CYH2, CYH1, URA3, bacterial and fungi toxins and the like can be used in reverse two-hybrid systems.

The polypeptides encoded by the nucleotide inserts of the rat's brain posterior bloc cells prey library thus prepared are termed “prey polypeptides” in the context of the presently described selection method of the prey polynucleotides.

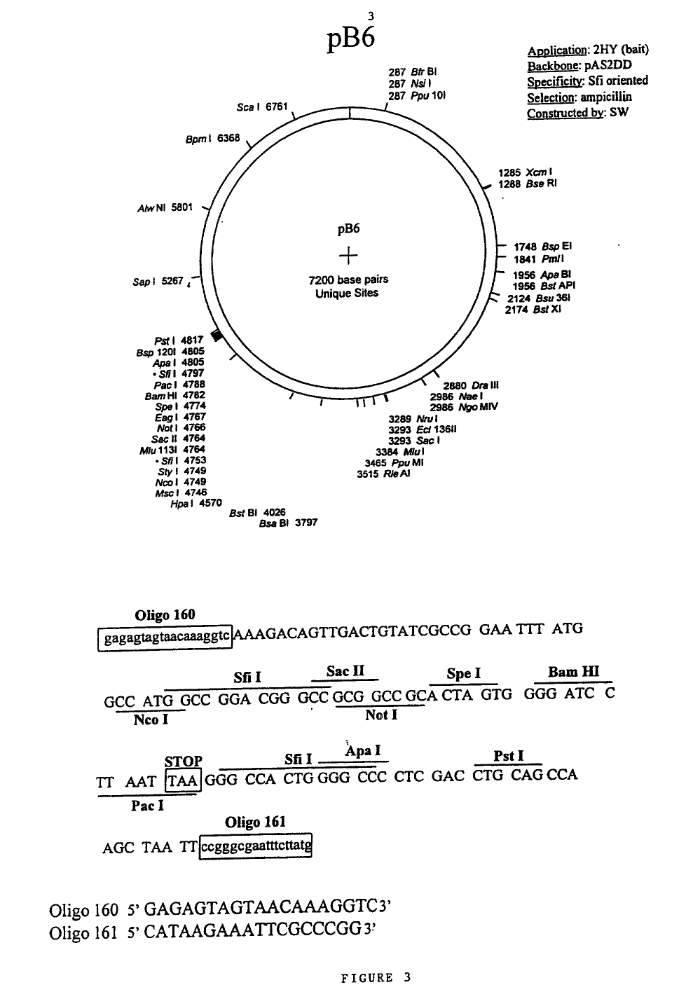

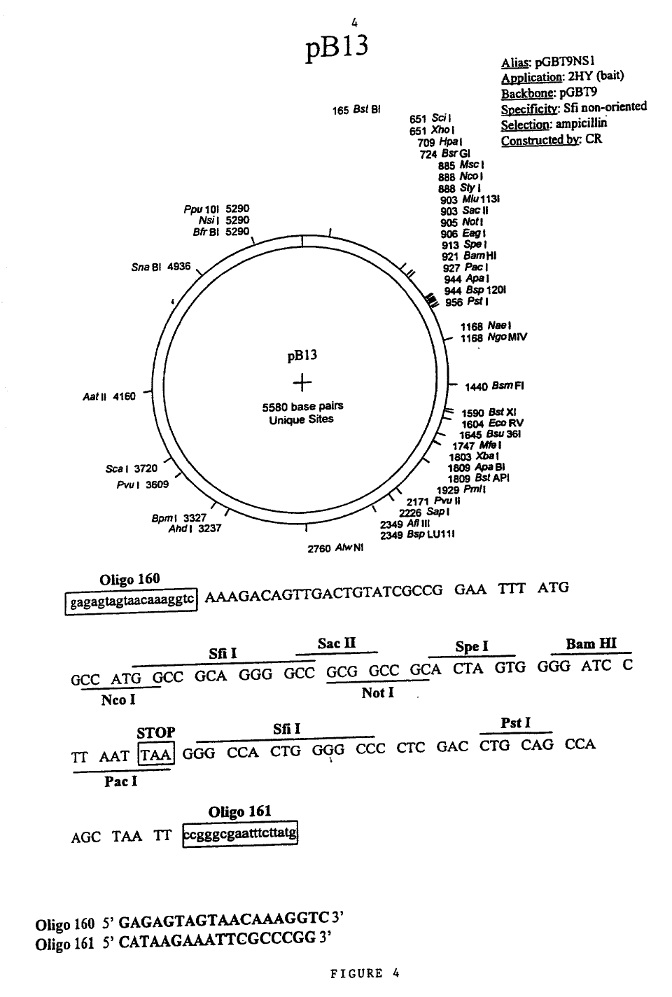

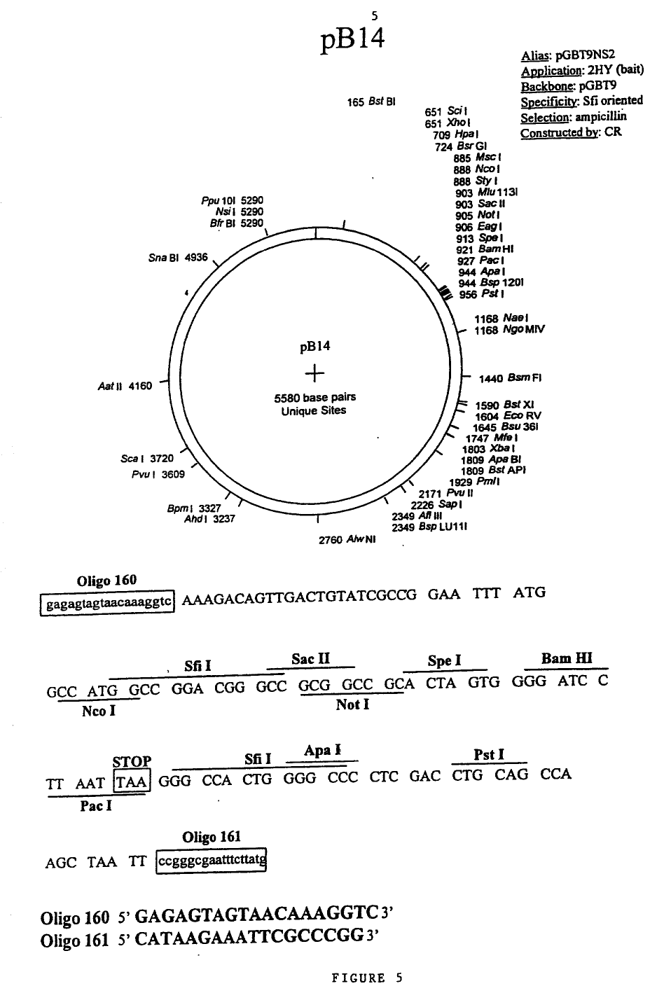

The bait polynucleotides can be inserted in bait plasmid pB6 as illustrated in FIG. 3. The bait polynucleotide insert is fused to a polynucleotide encoding the binding domain of, for example, the Gal4 DNA binding domain and the shuttle expression vector is used to transform cells.

The bait polynucleotides used in the present invention are described in Table 1.

As stated above, any cells can be utilized in transforming the bait and prey polynucleotides of the present invention including mammalian cells, bacterial cells, yeast cells, insect cells and the like.

In an embodiment, the present invention identifies protein-protein interactions in yeast. In using known methods a prey positive clone is identified containing a vector which comprises a nucleic acid insert encoding a prey polypeptide which binds to a bait polypeptide of interest. The method in which protein-protein interactions are identified comprises the following steps:

i) mating at least one first haploid recombinant yeast cell clone from a recombinant yeast cell clone library that has been transformed with a plasmid containing the prey polynucleotide to be assayed with a second haploid recombinant yeast cell clone transformed with a plasmid containing a bait polynucleotide encoding for the bait polypeptide;

ii) cultivating diploid cell clones obtained in step i) on a selective medium; and

iii) selecting recombinant cell clones which grow on the selective medium.

This method may further comprise the step of:

iv) characterizing the prey polynucleotide contained in each recombinant cell clone which is selected in step iii).

In yet another embodiment of the present invention, in lieu of yeast, Escherichia coli is used in a bacterial two-hybrid system, which encompasses a similar principle to that described above for yeast, but does not involve mating for characterizing the prey polynucleotide.

In yet another embodiment of the present invention, mammalian cells and a method similar to that described above for yeast for characterizing the prey polynucleotide are used.

By performing the yeast, bacterial or mammalian two-hybrid system, it is possible to identify for one particular bait an interacting prey polypeptide. The prey polypeptide that has been selected by testing the library of preys in a screen using the two-hybrid, two plus one hybrid methods and the like, encodes the polypeptide interacting with the protein of interest.

The present invention is also directed, in a general aspect, to a complex of polypeptides, polynucleotides encoding the polypeptides composed of a bait polypeptide or bait polynucleotide encoding the bait polypeptide and a prey polypeptide or prey polynucleotide encoding the prey polypeptide capable of interacting with the bait polypeptide of interest. These complexes are identified in Table 2.

In another aspect, the present invention relates to a complex of polynucleotides consisting of a first polynucleotide, or a fragment thereof, encoding a prey polypeptide that interacts with a bait polypeptide and a second polynucleotide or a fragment thereof. This fragment has at least about 12 consecutive nucleotides, but can have between about 12 and about 5,000 consecutive nucleotides, or between about 12 and about 10,000 consecutive nucleotides or between about 12 and about 20,000 consecutive nucleotides.

The complexes of the two interacting listed in Table 2 and the sets of two polynucleotides encoding these polypeptides also form part of the present invention.

In yet another embodiment, the present invention relates to an isolated complex of at least two polypeptides encoded by two polynucleotides wherein said two polypeptides are associated in the complex by affinity binding and are depicted in columns 1 and 3 of Table 1.

In yet another embodiment, the present invention relates to an isolated complex comprising at least a polypeptide as described in column 1 of Table 2 and a polypeptide as described in column 3 of Table 2. The present invention is not limited to these polypeptide complexes alone but also includes the isolated complex of the two polypeptides in which fragments and/or homologous polypeptides exhibiting at least 95% sequence identity, as well as from about 96% sequence identity to about 99.999% sequence identity.

Besides the isolated complexes described above, nucleic acids coding for a Selected Interacting Domain (SID®) polypeptide or a variant thereof or any of the nucleic acids can be inserted into an expression vector which contains the necessary elements for the transcription and translation of the inserted protein-coding sequence. Such transcription elements include a regulatory region and a promoter. Thus, the nucleic acid which may encode a marker compound of the present invention is operably linked to a promoter in the expression vector. The expression vector may also include a replication origin.

A wide variety of host/expression vector combinations are employed in expressing the nucleic acids of the present invention. Useful expression vectors that can be used include, for example, segments of chromosomal, non-chromosomal and synthetic DNA sequences. Suitable vectors include, but are not limited to, derivatives of SV40 and pcDNA and known bacterial plasmids such as col EI, pCR1, pBR322, pMal-C2, pET, pGEX as described by Smith et al (1988), pMB9 and derivatives thereof, plasmids such as RP4, phage DNAs such as the numerous derivatives of phage I such as NM989, as well as other phage DNA such as M13 and filamentous single stranded phage DNA; yeast plasmids such as the 2 micron plasmid or derivatives of the 2m plasmid, as well as centomeric and integrative yeast shuttle vectors; vectors useful in eukaryotic cells such as vectors useful in insect or mammalian cells; vectors derived from combinations of plasmids and phage DNAs, such as plasmids that have been modified to employ phage DNA or the expression control sequences; and the like.

For example in a baculovirus expression system, both non-fusion transfer vectors, such as, but not limited to pVL941 (BamHI cloning site Summers, pVL1393, BamHI, SmaI, Xbal, EcoRI, NotI, XmaIII, BglII and PstI cloning sites; Invitrogen) pVL1392 (BglII, PstI, NotI, XmaIII, EcoRI, XbalI, SmaI and BamHI cloning site; Summers and Invitrogen) and pBlueBacIII (BamHI, BglII, PstI, NcoI and HindIII cloning site, with blue/white recombinant screening, Invitrogen), and fusion transfer vectors such as, but not limited to, pAc700 (BamHI and KpnI cloning sites, in which the BamHI recognition site begins with the initiation codon; Summers), pAc701 and pAc70-2 (same as pAc700, with different reading frames), pAc360 (BamHI cloning site 36 base pairs downstream of a polyhedrin initiation codon; Invitrogen (1995)) and pBlueBacHisA, B, C (three different reading frames with BamHI, BGlII, PstI, NcoI and HindIII cloning site, an N-terminal peptide for ProBond purification and blue/white recombinant screening of plaques; Invitrogen (220 ) can be used.

Mammalian expression vectors contemplated for use in the invention include vectors with inducible promoters, such as the dihydrofolate reductase promoters, any expression vector with a DHFR expression cassette or a DHFR/methotrexate co-amplification vector such as pED (PstI, SalI, SbaI, SmaI and EcoRI cloning sites, with the vector expressing both the cloned gene and DHFR; Kaufman, 1991). Alternatively a glutamine synthetase/methionine sulfoximine co-amplification vector, such as pEE14 (HindIII, XbalI, SmaI, SbaI, EcoRI and BclI cloning sites in which the vector expresses glutamine synthetase and the cloned gene; Celltech). A vector that directs episomal expression under the control of the Epstein Barr Virus (EBV) or nuclear antigen (EBNA) can be used such as pREP4 (BamHI, SfiI, XhoI, NotI, NheI, HindIII, NheI, PvuII and KpnI cloning sites, constitutive RSV-LTR promoter, hygromycin selectable marker; Invitrogen), pCEP4 (BamHI, SfiI, XhoI, NotI, NheI, HindIII, NheI, PvuII and KpnI cloning sites, constitutive hCMV immediate early gene promoter, hygromycin selectable marker; Invitrogen), pMEP4 (KpnI, PvuI, NheI, HindIII, NotI, XhoI, SfiI, BamHI cloning sites, inducible methallothionein IIa gene promoter, hygromycin selectable marker, Invitrogen), pREP8 (BamHI, XhoI, NotI, HindIII, NheI and KpnI cloning sites, RSV-LTR promoter, histidinol selectable marker; Invitrogen), pREP9 (KpnI, NheI, HindIII, NotI, XhoI, SfiI, BamHI cloning sites, RSV-LTR promoter, G418selectable marker; Invitrogen), and pEBVHis (RSV-LTR promoter, hygromycin selectable marker, N-terminal peptide purifiable via ProBond resin and cleaved by enterokinase; Invitrogen).

Selectable mammalian expression vectors for use in the invention include, but are not limited to, pRc/CMV (HindIII, BstXI, NotI, SbaI and ApaI cloning sites, G418selection, Invitrogen), pRc/RSV (HindII, SpeI, BstXI, NotI, XbaI cloning sites, G418selection, Invitrogen) and the like. Vaccinia virus mammalian expression vectors (see, for example Kaufman 1991 that can be used in the present invention include, but are not limited to, pSC11 (SmaI cloning site, TK- and β-gal selection), pMJ601 (SalI, SmaI, AflI, NarI, BspMII, BamHI, ApaI, NheI, SacII, KpnI and HindIII cloning sites; TK- and β-gal selection), pTKgptF1S (EcoRI, PstI, SalII, AccI, HindII, SbaI, BamHI and Hpa cloning sites, TK or XPRT selection) and the like.

Yeast expression systems that can also be used in the present include, but are not limited to, the non-fusion pYES2 vector (XbaI, SphI, ShoI, NotI, GstXI, EcoRI, BstXI, BamHI, SacI, KpnI and HindIII cloning sites, Invitrogen), the fusion pYESHisA, B, C (XbalI, SphI, ShoI, NotI, BstXI, EcoRI, BamHI, SacI, KpnI and HindIII cloning sites, N-terminal peptide purified with ProBond resin and cleaved with enterokinase; Invitrogen), pRS vectors and the like.

Consequently, mammalian and typically human cells, as well as bacterial, yeast, fungi, insect, nematode and plant cells an used in the present invention and may be transfected by the nucleic acid or recombinant vector as defined herein.

Examples of suitable cells include, but are not limited to, VERO cells, HELA cells such as ATCC No. CCL2, CHO cell lines such as ATCC No. CCL61, COS cells such as COS-7 cells and ATCC No. CRL 1650 cells, W138, BHK, HepG2, 3T3 such as ATCC No. CRL6361, A549, PC12, K562 cells, 293 cells, Sf9 cells such as ATCC No. CRL1711 and Cv1 cells such as ATCC No. CCL70.

Other suitable cells that can be used in the present invention include, but are not limited to, prokaryotic host cells strains such as Escherichia coli, (e.g., strain DH5α), Bacillus subtilis, Salmonella typhimurium, or strains of the genera of Pseudomonas, Streptomyces and Staphylococcus.

Further suitable cells that can be used in the present invention include yeast cells such as those of Saccharomyces such as Saccharomyces cerevisiae.

Besides the specific isolated complexes, as described above, the present invention relates to and also encompasses SID® polynucleotides. As explained above, for each bait polypeptide, several prey polypeptides may be identified by comparing and selecting the intersection of every isolated fragment that are included in the same polypeptide. Thus the SID® polynucleotides of the present invention are represented by the shared nucleic acid sequences of SEQ ID Nos. 5 and 6 encoding the SID® polypeptides of SEQ ID Nos. 7 and 8 in column 7 of Table 3.

The present invention is not limited to the SID® sequences as described in the above paragraph, but also includes fragments of these sequences having at least 12 consecutive nucleic acids, between about 12 and about 5,000 consecutive nucleic acids and between about 12 and about 10,000 consecutive nucleic acids and between about 12 and about 20,000 consecutive nucleic acids, as well as variants thereof. The fragments or variants of the SID® sequences possess at least the same affinity of binding to its protein or polypeptide counterpart, against which it has been initially selected. Moreover this variant and/or fragments of the SID® sequences alternatively can have between 95% and 99.999% sequence identity to its protein or polypeptide counterpart.

According to the present invention variants of polynucleotide or polypeptides can be created by known mutagenesis techniques either in vitro or in vivo. Such a variant can be created such that it has altered binding characteristics with respect to the target protein and more specifically that the variant binds the target sequence with either higher or lower affinity.

Polynucleotides that are complementary to the above sequences which include the polynucleotides of the SID®'s, their fragments, variants and those that have specific sequence identity are also included in the present invention.

The polynucleotide encoding the SID® polypeptide, fragment or variant thereof can also be inserted into recombinant vectors which are described in detail above.

The present invention also relates to a composition comprising the above-mentioned recombinant vectors containing the SID® polypeptides in Table 3, fragments or variants thereof, as well as recombinant host cells transformed by the vectors. The recombinant host cells that can be used in the present invention were discussed in greater detail above.

The compositions comprising the recombinant vectors can contain physiological acceptable carriers such as diluents, adjuvants, excipients and any vehicle in which this composition can be delivered therapeutically and can include, but is are not limited to sterile liquids such as water and oils.

According to the present invention variants of polynucleotide or polypeptides can be created by known mutagenesis techniques either in vitro or in vivo. Such a variant can be created such that it has altered binding characteristics with respect to the target protein and more specifically that the variant binds the target sequence with either higher or lower affinity.

The compositions comprising the recombinant vectors can contain physiological acceptable carriers such as diluents, adjuvants, excipients and any vehicle in which this composition can be delivered therapeutically and can include, but is are not limited to sterile liquids such as water and oils.

In yet another embodiment, the present invention relates to a method of selecting modulating compounds, as well as the modulating molecules or compounds themselves which may be used in a pharmaceutical composition. These modulating compounds may act as a cofactor, as an inhibitor, as antibodies, as tags, as a competitive inhibitor, as an activator or alternatively have agonistic or antagonistic activity on the protein-protein interactions.

The activity of the modulating compound does not necessarily, for example, have to be 100% activation or inhibition. Indeed, even partial activation or inhibition can be achieved that is of pharmaceutical interest.

The modulating compound can be selected according to a method which comprises:

(a) cultivating a recombinant host cell with a modulating compound on a selective medium and a reporter gene the expression of which is toxic for said recombinant host cell wherein said recombinant host cell is transformed with two vectors:

(i) wherein said first vector comprises a polynucleotide encoding a first hybrid polypeptide having a DNA binding domain;

(ii) wherein said second vector comprises a polynucleotide encoding a second hybrid polypeptide having a transcriptional activating domain that activates said toxic reporter gene when the first and second hybrid polypeptides interact;

(b) selecting said modulating compound which inhibits or permits the growth of said recombinant host cell.

Thus, the present invention relates to a modulating compound that inhibits the protein-protein interactions of a complex of two polypeptides of columns 1 and 3 of Table 2. The present invention also relates to a modulating compound that activates the protein-protein interactions of a complex of two polypeptides of columns 1 and 3 of Table 2.

In yet another embodiment, the present invention relates to a method of selecting a modulating compound, which modulating compound inhibits the interactions of two polypeptides of columns 1 and 3 of Table 2. This method comprises:

(a) cultivating a recombinant host cell with a modulating compound on a selective medium and a reporter gene the expression of which is toxic for said recombinant host cell wherein said recombinant host cell is transformed with two vectors:

(i) wherein said first vector comprises a polynucleotide encoding a first hybrid polypeptide having a first domain of an enzyme;

(ii) wherein said second vector comprises a polynucleotide encoding a second hybrid polypeptide having an enzymatic transcriptional activating domain that activates said toxic reporter gene when the first and second hybrid polypeptides interact;

(b) selecting said modulating compound which inhibits or permits the growth of said recombinant host cell.

In the two methods described above any toxic reporter gene can be utilized including those reporter genes that can be used for negative selection including the URA3 gene, the CYH1 gene, the CYH2 gene and the like.

In yet another embodiment, the present invention provides a kit for screening a modulating compound. This kit comprises a recombinant host cell which comprises a reporter gene the expression of which is toxic for the recombinant host cell. The host cell is transformed with two vectors. The first vector comprises a polynucleotide encoding a first hybrid polypeptide having a DNA binding domain; and a second vector comprises a polynucleotide encoding a second hybrid polypeptide having a transcriptional activating domain that activates said toxic reporter gene when the first and second hybrid polypeptides interact.

In yet another embodiment, a kit is provided for screening a modulating compound by providing a recombinant host cell, as described in the paragraph above, but instead of a DNA binding domain, the first vector comprises a first hybrid polypeptide containing a first domain of a protein. The second vector comprises a second polypeptide containing a second part of a complementary domain of a protein that activates the toxic reporter gene when the first and second hybrid polypeptides interact.

In the selection methods described above, the activating domain can be p42 Gal 4, YP16 (HSV) and the DNA-binding domain can be derived from Gal4 or Lex A. The protein or enzyme can be adenylate cyclase, guanylate cyclase, DHFR and the like.

In yet another embodiment, the present invention relates to a pharmaceutical composition comprising the modulating compounds for preventing or treating deafness and hearing disorders and/or diseases in a human or animal, most preferably in a mammal.

This pharmaceutical composition comprises a pharmaceutically acceptable amount of the modulating compound. The pharmaceutically acceptable amount can be estimated from cell culture assays. For example, a dose can be formulated in animal models to achieve a circulating concentration range that includes or encompasses a concentration point or range having the desired effect in an in vitro system. This information can thus be used to accurately determine the doses in other mammals, including humans and animals.

The therapeutically effective dose refers to that amount of the compound that results in amelioration of symptoms in a patient. Toxicity and therapeutic efficacy of such compounds can be determined by standard pharmaceutical procedures in cell cultures or in experimental animals. For example, the LD50 (the dose lethal to 50% of the population) as well as the ED50 (the dose therapeutically effective in 50% of the population) can be determined using methods known in the art. The dose ratio between toxic and therapeutic effects is the therapeutic index which can be expressed as the ratio between LD 50 and ED50 compounds that exhibit high therapeutic indexes.

The data obtained from the cell culture and animal studies can be used in formulating a range of dosage of such compounds which lies preferably within a range of circulating concentrations that include the ED50 with little or no toxicity.

The pharmaceutical composition can be administered via any route such as locally, orally, systemically, intravenously, intramuscularly, mucosally, using a patch and can be encapsulated in liposomes, microparticles, microcapsules, and the like. The pharmaceutical composition can be embedded in liposomes or even encapsulated.

Any pharmaceutically acceptable carrier or adjuvant can be used in the pharmaceutical composition. The modulating compound will be preferably in a soluble form combined with a pharmaceutically acceptable carrier. The techniques for formulating and administering these compounds can be found in “Remington's Pharmaceutical Sciences” Mack Publication Co., Easton, Pa., latest edition.

In yet another embodiment, the present invention relates to a pharmaceutical composition comprising a SID® polypeptide, a fragment or variant thereof. The SID® polypeptide, fragment or variant thereof can be used in a pharmaceutical composition provided that it is endowed with highly specific binding properties to a bait polypeptide of interest.

The original properties of the SID® polypeptide or variants thereof interfere with the naturally occurring interaction between a first protein and a second protein within the cells of the organism. Thus, the SID® polypeptide binds specifically to either the first polypeptide or the second polypeptide.

Therefore, the SID® polypeptides of the present invention or variants thereof interfere with protein-protein interactions between inner ear proteins.

Thus, the present invention relates to a pharmaceutical composition comprising a pharmaceutically acceptable amount of a SID® polypeptide or variant thereof, provided that the variant has the above-mentioned two characteristics; i.e., that it is endowed with highly specific binding properties to a bait polypeptide of interest and is devoid of biological activity of the naturally occurring protein.

In yet another embodiment, the present invention relates to a pharmaceutical composition comprising a pharmaceutically effective amount of a polynucleotide encoding a SID® polypeptide or a variant thereof wherein the polynucleotide is placed under the control of an appropriate regulatory sequence. Appropriate regulatory sequences that are used are polynucleotide sequences derived from promoter elements and the like.

Polynucleotides that can be used in the pharmaceutical composition of the present invention include the nucleotide sequences of SEQ ID Nos. 5 and 6.

Besides the SID® polypeptides and polynucleotides, the pharmaceutical composition of the present invention can also include a recombinant expression vector comprising the polynucleotide encoding the SID® polypeptide, fragment or variant thereof.

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

The SID® polypeptides as active ingredients will be preferably in a soluble form combined with a pharmaceutically acceptable carrier. The techniques for formulating and administering these compounds can be found in “Remington's Pharmaceutical Sciences” supra.

The amount of pharmaceutically acceptable SID® polypeptides can be determined as described above for the modulating compounds using cell culture and animal models.

Such compounds can be used in a pharmaceutical composition to treat or prevent hearing disorders and/or diseases.

Thus, the present invention also relates to a method of preventing or treating hearing disorders and/or diseases in a mammal said method comprising the steps of administering to a mammal in need of such treatment a pharmaceutically effective amount of:

(1) a SID® polypeptide of SEQ ID Nos. 5 and 6 or a variant thereof which binds to a targeted protein; or

(2) or SID® polynucleotide encoding a SID® polypeptide of SEQ ID Nos. 7 and 8 or a variant or a fragment thereof wherein said polynucleotide is placed under the control of a regulatory sequence which is functional in said mammal; or

(3) a recombinant expression vector comprising a polynucleotide encoding a SID® polypeptide which binds to an inner ear protein.

In another embodiment the present invention nucleic acids comprising a sequence of SEQ ID Nos. 1 and 2 which encodes the protein of sequence SEQ ID Nos. 3 and 4 and/or functional derivatives thereof are administered to modulate complex (from Table 2) function by way of gene therapy. Any of the methodologies relating to gene therapy available within the art may be used in the practice of the present invention such as those described by Goldspiel et al Clin. Pharm. 12 pgs. 488-505 (1993).

The mode of administration optimum dosages and galenic forms can be determined by the criteria known in the art taken into account the seriousness of the general condition of the mammal, the tolerance of the treatment and the side effects.

The present invention also relates to a method of treating or preventing inner ear diseases in a human or mammal in need of such treatment. This method comprises administering to a mammal in need of such treatment a pharmaceutically effective amount of a modulating compound which binds to a targeted mammalian or human or inner ear cell protein. In a preferred embodiment, the modulating compound is a polynucleotide which may be placed under the control of a regulatory sequence which is functional in the mammal or human.

The above described pharmaceutical compositions can be administered by any route such as orally, systemically, intravenously, intramuscularly, intradermally, mucosally, encapsulated, using a patch and the like. Any pharmaceutically acceptable carrier or adjuvant can be used in this pharmaceutical composition.

Delivery of the therapeutic nucleic acid into a patient may be direct in vivo gene therapy (i.e., the patient is directly exposed to the nucleic acid or nucleic acid-containing vector) or indirect ex vivo gene therapy (i.e., cells are first transformed with the nucleic acid in vitro and then transplanted into the patient).

For example for in vivo gene therapy, an expression vector containing the nucleic acid is administered in such a manner that it becomes intracellular; i.e., by infection using a defective or attenuated retroviral or other viral vectors as described, for example in U.S. Pat. No. 4,980,286 or by Robbins et al, Pharmacol. Ther., 80 No. 1 pgs. 35-47 (1998).

The various retroviral vectors that are known in the art are such as those described in Miller et al. (Meth. Enzymol. 217 pgs. 581-599 (1993)) which have been modified to delete those retroviral sequences which are not required for packaging of the viral genome and subsequent integration into host cell DNA. Also adenoviral vectors can be used which are advantageous due to their ability to infect non-dividing cells and such high-capacity adenoviral vectors are described in Kochanek (Human Gene Therapy, 10, pgs. 2451-2459 (1999)). Chimeric viral vectors that can be used are those described by Reynolds et al. (Molecular Medecine Today, pgs. 25-31 (1999)). Hybrid vectors can also be used and are described by Jacoby et al. (Gene Therapy, 4, pgs. 1282-1283 (1997)).

Direct injection of naked DNA or through the use of microparticle bombardment (e.g., Gene Gun®; Biolistic, Dupont) or by coating it with lipids can also be used in gene therapy. Cell-surface receptors/transfecting agents or through encapsulation in liposomes, microparticles or microcapsules or by administering the nucleic acid in linkage to a peptide which is known to enter the nucleus or by administering it in linkage to a ligand predisposed to receptor-mediated endocytosis (See Wu & Wu, J. Biol. Chem., 262 pgs. 4429-4432 (1987)) can be used to target cell types which specifically express the receptors of interest.

In another embodiment a nucleic acid ligand compound may be produced in which the ligand comprises a fusogenic viral peptide designed so as to disrupt endosomes, thus allowing the nucleic acid to avoid subsequent lysosomal degradation. The nucleic acid may be targeted in vivo for cell specific endocytosis and expression by targeting a specific receptor such as that described in WO92/06180, WO93/14188 and WO 93/20221. Alternatively the nucleic acid may be introduced intracellularly and incorporated within the host cell genome for expression by homologous recombination (See Zijlstra et al, Nature, 342, pgs. 435-428 (1989)).

In ex vivo gene therapy, a gene is transferred into cells in vitro using tissue culture and the cells are delivered to the patient by various methods such as injecting subcutaneously, application of the cells into a skin graft and the intravenous injection of recombinant blood cells such as hematopoietic stem or progenitor cells.

Cells into which a nucleic acid can be introduced for the purposes of gene therapy include, for example, epithelial cells, endothelial cells, keratinocytes, fibroblasts, muscle cells, hepatocytes and blood cells. The blood cells that can be used include, for example, T-lymphocytes, B-lymphocytes, monocytes, macrophages, neutrophils, eosinophils, megakaryotcytes, granulocytes, hematopoietic cells or progenitor cells and the like.

In yet another embodiment the present invention relates to protein chips or protein microarrays. It is well known in the art that microarrays can contain more than 10,000 spots of a protein that can be robotically deposited on a surface of a glass slide or nylon filter. The proteins attach covalently to the slide surface, yet retain their ability to interact with other proteins or small molecules in solution. In some instances the protein samples can be made to adhere to glass slides by coating the slides with an aldehyde-containing reagent that attaches to primary amines. A process for creating microarrays is described, for example by MacBeath and Schreiber (Science, Volume 289, Number 5485, pgs, 1760-1763 (2000)) or (Service, Science, Vol, 289, Number 5485 pg. 1673 (2000)). An apparatus for controlling, dispensing and measuring small quantities of fluid is described, for example, in U.S. Pat. No. 6,112,605.

The present invention also provides a record of protein-protein interactions, PIM®'s and any data encompassed in the following Tables. It will be appreciated that this record can be provided in paper or electronic or digital form.

It has been concluded that an early inter-stereocilial adhesion process is required to shape a coherent hair bundle. It relies on myosin VIIa, harmonin b and cadherin 23 acting together as a functional network.

More specifically, as exemplified in greater detail, this conclusion was in fact reached by using the following general procedure, which is disclosed in greater detail in the examples below. The llibrary was constructed as follows. A total of 292 mice, aged from P0 to P10 were dissected and only the vestibular sensory epithelia were used to generate a random-primed cDNA library into the pP6 plasmid (Rain et al , Nature 2001 409:211-215). The complexity of the primary library was over 50 million clones. Sequence analysis was performed on two hundred randomly chosen clones to establish the characteristics of the library. The library was then transformed into yeast and ten million independent yeast colonies were collected, pooled and stored at −80° C. as equivalent aliquot fractions of the same library.

Screening was performed using two different baits, namely the C-terminal fragment of myosin VIIa tail (amino acids 1562 to 2215), composed of SH3, MyTH4 and FERM domains, and the intracellular region of cadherin 23 (amino acids 3086 to 3354). Both baits were cloned in the plasmid pB6 derived from the original pAS2ΔΔ (Fromont-Racine M. et al, Nature Genetics. 1997, 16: 277-282) and described in greater detail below in the Examples.

The mating protocol that was used is described in more detail below in the examples.

Briefly, the screening conditions were adapted for each bait (test screen) before performing the full-size screening. The selectivity of the HIS3 reporter gene was eventually modulated with aminotriazole in order to obtain a maximum of 384 histidine-positive clones. For all the selected clones, LacZ activity was measured in a semi-quantitative X-Gal overlay assay.

The prey fragments of the positive clones were amplified by PCR, analysed on agarose gel and sequences at their 5′ and 3′ junctions on a Perkin Elmer 3700 Sequencer. The resulting sequences were then used to identify the corresponding gene in the GenBank data base (NCBI) using a fully automated procedure.

The expression constructs used were the following. A cDNA encoding the cytoplasmic region of the human cadherin 23 (NM-022124; amino acids 3086 to 3354) was obtained by RACE-PCR using the inner ear cDNA library. PCR products were subcloned into pCMV-tag3B (Myc tag, Stratagene) and pcDNA (No tag, Invitrogen) for expression in HeLa cells and into pGex-4T1 (GST tag Amersham) and pXa3 (Biotin tag, Promega) for protein production.

The mouse full-length cDNAs encoding harmonin isoforms al (AF228924; amino acids 1 to 548), b2 (AY103465; amino acids 1 to 859) and cl (amino acids 1 to 403) were amplified using the inner ear cDNA library as a template and cloned into pCMV-tag3C(Stratagene), pcDNA (Invitrogen) and in pGEX-4T1 (Amersham). cDNAs encoding harmonin truncated forms, i.e., PDZ-1 PDZ2 (amino acids 72 to 307), PDZ1(amino acids 72 to 88), PDZ2 (amino acids 189 to 307) and PDZ3 (amino acids 738 to 849) were subcloned into pCMV-tag3C (Strategene) and pGEX4T1 (Amersham).

A human cDNA encoding the myosin VIIa tail; (amino acids 847 to 2,215) was cloned in pcDNA (Strategene) for expression in HeLa cells. For protein production a zebrafish cDNA encoding his-tagged myosin VIIa C-terminal tail fragment (88.5% amino acids identity with the corresponding human fragment) was subcloned in the pFastBac HTa vector (baculovirus, Life Technologies).

The antibody production, pull down and in vitro binding experiments, immunofluorescence and electron microscopy analysis, BAPTA and subtilisin treatment of mouse cochlear cultures and the actin bundling and cosedimenttaion assays are described in greater detail below in the examples.

fully illustrate the present invention and advantages thereof, the following specific examples are given, it being understood that the same are intended only as illustrative and in nowise limitative.

### EXAMPLES

Animals

In the examples that follow the following animals were used. Wistar rats and RJ swiss mice (Janvier, France) were used. Embryonic day 0 (Eo) was determined by vaginal plug detection and the day of birth was P0. The original spontaneous shaker-1 (Myo7a^{sh1}) mutant was obtained from Jackson laboratories (Bar Harbor, USA). The shaker-1Myo7a^{4626SB} allele, obtained by ENU-mutagenesis, was kindly provided by Dr. K. Steel (MRC, U.K.). For shaker-1 mice genotyping, P12 mice or older were classified as homozygous mutants or heterozygous controls on the basis of their hyperactivity and circling behaviour. Younger mice were genotyped as described by Self et al, Development 125, 557-66 (1998)). The absence of myosin VIIa in the Myo7a^{4626SB} mice was confirmed using the anti-myosin VIIa antibody. The following mice were studied: Myo7a^{sh1} stock mutants at E20, P2, P6, P10 and P25. Myo7a^{4626SB} stock, 15 mutants at E20, P0, P2, P6, P10, P25, P30 and P60, all with the same number of littermate controls.

### Example 1

Preparation of a Collection of Random-Primed cDNA Fragments

1.A. Collection Preparation and Transformation in Escherichia coli

1.A.1. Random-Primed cDNA Fragment Preparation

For mRNA sample from mousers inner ear cells, random-primed cDNA was prepared from 5 μg of polyA+ mRNA using a TimeSaver cDNA Synthesis Kit (Amersham Pharmacia Biotech) and with 5 μg of random N9-mers according to the manufacturer's instructions. Following phenolic extraction, the cDNA was precipitated and resuspended in water. The resuspended cDNA was phosphorylated by incubating in the presence of T4 DNA Kinase (Biolabs) and ATP for 30 minutes at 37° C. The resulting phosphorylated cDNA was then purified over a separation column (Chromaspin TE 400, Clontech), according to the manufacturer's protocol.

1.A.2. Ligation of Linkers to Blunt-Ended cDNA

Oligonucleotide HGX931 (5′ end phosphorylated) 1 μg/μl and HGX932 1 μg/μl.

Sequence of the oligo HGX931: 5′-GGGCCACGAA-3′ (SEQ ID No. 9)

Sequence of the oligo HGX932: 5′-TTCGTGGCCCCTG-3′ (SEQ ID No. 10)

Linkers were preincubated (5 minutes at 95° C., 10 minutes at 68° C., 15 minutes at 42° C.) then cooled down at room temperature and ligated with cDNA fragments at 16° C. overnight.

Linkers were removed on a separation column (Chromaspin TE 400, Clontech), according to the manufacturer's protocol.

1.A.3. Vector Preparation

Plasmid pP6 (see FIG. 10) was prepared by replacing the SpeI/XhoI fragment of pGAD3S2X with the double-stranded oligonucleotide: 5′-CTAGCCATGGCCGCAGGGGCCGCGGCCGCACTAGTGGGGATCCTTAATTAAAGGGCCACTG GGGCCCCCGGTACCGGCGTCCCCGGCGCCGGCGTGATCACCCCTAGGAATTAATTTCCCGG TGACCCCGGGGGAGCT-3′ (SEQ ID No. 11)

The pP6 vector was successively digested with Sfi1 and BamHI restriction enzymes (Biolabs) for 1 hour at 37° C., extracted, precipitated and resuspended in water. Digested plasmid vector backbones were purified on a separation column (Chromaspin TE 400, Clontech), according to the manufacturer's protocol.

1.A.4. Ligation Between Vector and Insert of cDNA

The prepared vector was ligated overnight at 15° C. with the blunt-ended cDNA described in section 2 using T4 DNA ligase (Biolabs). The DNA was then precipitated and resuspended in water.

1.A.5. Library Transformation in Escherichia coli

The DNA from section 1.A.4 was transformed into Electromax DH10B electrocompetent cells (Gibco BRL) with a Cell Porator apparatus (Gibco BRL). 1 ml SOC medium was added and the transformed cells were incubated at 37° C. for 1 hour. 9 mls of SOC medium per tube was added and the cells were plated on LB+ampicillin medium. The colonies were scraped with liquid LB medium, aliquoted and frozen at −80° C.

The obtained collection of recombinant cell clones was named HGXBMIERP1.

1.B. Collection Transformation in Saccharomyces cerevisiae

The Saccharomyces cerevisiae strain (Y187 (MATα Gal4Δ Gal80Δ ade2-101, his3, leu2-3, -112, trp1-901, ura3-52 URA3::UASGAL1-LacZ Met)) was transformed with the cDNA library.

The plasmid DNA contained in E. coli were extracted (Qiagen) from aliquoted E. coli frozen cells (1.A.5.). Saccharomyces cerevisiae yeast Y187 in YPGlu were grown.

Yeast transformation was performed according to standard protocol (Giest et al. Yeast, 11, 355-360, 1995) using yeast carrier DNA (Clontech). This experiment leads to 10⁴ to 5×10⁴ cells/μg DNA. 2×10⁴ cells were spread on DO-Leu medium per plate. The cells were aliquoted into vials containing 1 ml of cells and frozen at −80° C.

The obtained collection of recombinant cell clones was named HGXYMIERP1.

1.C. Construction of Bait Plasmids

For fusions of the bait protein to the DNA-binding domain of the GAL4 protein of S. cerevisiae, bait fragments were cloned into plasmid pB6 or plasmid pB27.

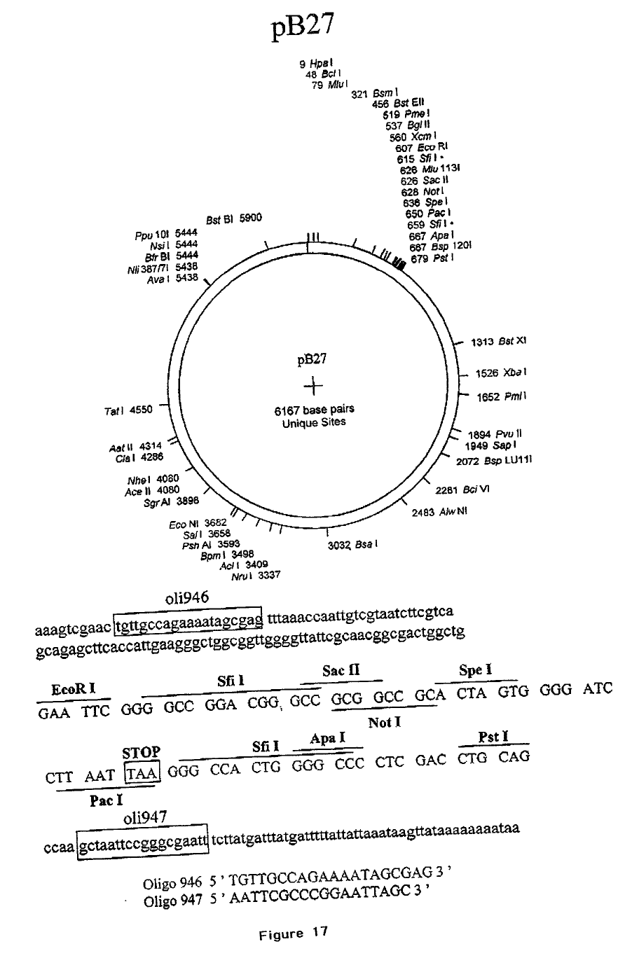

Plasmid pB6 (see FIG. 3) or pB27 (see FIG. 17) was prepared by replacing the Nco1/Sal1 polylinker fragment of pASΔΔ with the double-stranded DNA fragment:

5′ CATGGCCGGACGGGCCGCGGCCGCACTAGTGGGGATCCTTAATTAAAGG GCCACTGGGGCCCCC 3′ (SEQ ID No. 12)

3′ CGGCCTGCCCGGCGCCGGCGTGATCACCCCTAGGAATTAATTTCCCGGT GACCCCGGGGGAGCT 5′ (SEQ ID No. 13)

The amplification of the bait ORF was obtained by PCR using the Pfu proof-reading Taq polymerase (Stratagene), 10 pmol of each specific amplification primer and 200 ng of plasmid DNA as template.

The PCR program was set up as follows:

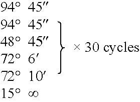

The amplification was checked by agarose gel electrophoresis.

The PCR fragments were purified with Qiaquick column (Qiagen) according to the manufacturer's protocol.

Purified PCR fragments were digested with adequate restriction enzymes.

The PCR fragments were purified with Qiaquick column (Qiagen) according to the manufacturer's protocol.

The digested PCR fragments were ligated into an adequately digested and dephosphorylated bait vector (pB6 or pB27) according to standard protocol (Sambrook et al.) and were transformed into competent bacterial cells. The cells were grown, the DNA extracted and the plasmid was sequenced.

### Example 2

Screening the Collection with the Two-Hybrid in Yeast System

2.A. The Mating Protocol

The mating two-hybrid in yeast system (as described by Legrain et al., Nature Genetics, vol. 16, 277-282 (1997), Toward a functional analysis of the yeast genome through exhaustive two-hybrid screens) was used for its advantages but one could also screen the cDNA collection in classical two-hybrid system as described in Fields et al. or in a yeast reverse two-hybrid system.

The mating procedure allows a direct selection on selective plates because the two fusion proteins are already produced in the parental cells. No replica plating is required.

This protocol was written for the use of the library transformed into the Y187 strain.

For bait proteins fused to the DNA-binding domain of GAL4, bait-encoding plasmids were first transformed into S. cerevisiae (CG1945 strain (MATa Gal4-542 Gal180-538 ade2-101 his3Δ200, leu2-3, 112, trp1-901, ura3-52, lys2-801, URA3: :GAL4 17mers (X3)-CyC1TATA-LacZ, LYS2: :GAL1UAS-GAL1TATA-HIS3 CYH^{R})) according to step 1.B. and spread on DO-Trp medium.

For bait proteins fused to the DNA-binding domain of LexA, bait-encoding plasmids were first transformed into S. cerevisiae (L40Δgal4 strain (MATa ade2, trp1-901, leu2 3,112, lys2-801, his3Δ200, LYS2:: (lexAop)₄-HIS3, ura3-52::URA3 (lexAop)₈-LacZ, GAL4::Kan^{R})) according to step 1.B. and spread on DO-Trp medium.

Day 1, Morning: Preculture

The cells carrying the bait plasmid obtained at step 1.C. were precultured in 20 ml DO-Trp medium and grown at 30° C. with vigorous agitation.

Day 1, Late Afternoon: Culture

The OD₆₀₀ₙₘ of the DO-Trp pre-culture of cells carrying the bait plasmid pre-culture was measured. The OD₆₀₀ₙₘ must lie between 0.1 and 0.5 in order to correspond to a linear measurement.

50 ml DO-Trp at OD₆₀₀ₙₘ 0.006/ml was inoculated and grown overnight at 30° C. with vigorous agitation.

Day 2: Mating

Medium and Plates

1 YPGlu 15 cm plate

50 ml tube with 13 ml DO-Leu-Trp-His

100 ml flask with 5 ml of YPGlu

8 DO-Leu-Trp-His plates

2 DO-Leu plates

2DO-Trp plates

2 DO-Leu-Trp plates

The OD₆₀₀ₙₘ of the DO-Trp culture was measured. It should be around 1.

For the mating, twice as many bait cells as library cells were used. To get a good mating efficiency, one must collect the cells at 10 cells per cm².

The amount of bait culture (in ml) that makes up 50 OD₆₀₀ₙₘ units for the mating with the prey library was estimated.

A vial containing the HGXYMIERP1 library was thawed slowly on ice. 1.0 ml of the vial was added to 5 ml YPGlu. Those cells were recovered at 300° C., under gentle agitation for 10 minutes.

Mating

The 50 OD₆₀₀ₙₘ units of bait culture was placed into a 50 ml falcon tube.

The HGXYMIERP1 library culture was added to the bait culture, then centrifuged, the supernatant discarded and resuspended in 1.6 ml YPGlu medium.

The cells were distributed onto two 15 cm YPGlu plates with glass beads. The cells were spread by shaking the plates. The plate cells-up at 30° C. for 4 h 30 min were incubated.

Collection of Mated Cells

The plates were washed and rinsed with 6 ml and 7 ml respectively of DO-Leu-Trp-His. Two parallel serial ten-fold dilutions were performed in 500 μl DO-Leu-Trp-His up to 1/10,000. 50 μL of each 1/10000 dilution was spread onto DO-Leu and DO-trp plates and 50 μl of each 1/1000 dilution onto DO-Leu-Trp plates. 22.4 ml of collected cells were spread in 400 μl aliquots on DO-Leu-Trp-His+Tet plates.

Day 4

Clones that were able to grow on DO-Leu-Trp-His+Tetracyclin were then selected. This medium allows one to isolate diploid clones presenting an interaction.

The His+ colonies were counted on control plates.

The number of His+ cell clones will define which protocol is to be processed:

Upon 60.10⁶ Trp+Leu+ colonies

if the number His+ cell clones <285: then use the process luminometry protocol on all colonies

if the number of His+ cell clones >285 and <5000: then process via overlay and then luminometry protocols on blue colonies (2.B and 2.C).

if number of His+ cell clones >5000: repeat screen using DO-Leu-Trp-His+ Tetracyclin plates containing 3-aminotriazol.

2.B. The X-Gal overlay assay

The X-Gal overlay assay was performed directly on the selective medium plates after scoring the number of His⁺ colonies.

Materials

A water bath was set up. The water temperature should be 50° C.

0.5 M Na₂HPO₄ pH 7.5.

1.2% Bacto-agar.

2% X-Gal in DMF.

Overlay mixture: 0.25 M Na₂HPO₄ pH7.5, 0.5% agar, 0.1% SDS, 7% DMF (LABOSI), 0.04% X-Gal (ICN). For each plate, 10 ml overlay mixture are needed.

DO-Leu-Trp-His plates.

Sterile toothpicks.

Experiment

The temperature of the overlay mix should be between 45° C. and 50° C. The overlay-mix was poured over the plates in portions of 10 ml. When the top layer was settled, they were collected. The plates were incubated overlay-up at 30° C. and the time was noted. Blue colonies were checked for regularly. If no blue colony appeared, overnight incubation was performed. Using a pen the number of positives was marked. The positives colonies were streaked on fresh DO-Leu-Trp-His plates with a sterile toothpick.

2.C. The Luminometry Assay

His+ colonies were grown overnight at 30° C. in microtiter plates containing DO-Leu-Trp-His+Tetracyclin medium with shaking. The day after, the overnight culture was diluted 15 times into a new microtiter plate containing the same medium and was incubated for 5 hours at 30° C. with shaking. The samples were diluted 5 times and read OD₆₀₀ₙₘ. The samples were diluted again to obtain between 10,000 and 75,000 yeast cells/well in 100 μl final volume.

Per well, 76 μl of One Step Yeast Lysis Buffer (Tropix) was added, 20 μl SapphireII Enhancer (Tropix), 4 μl Galacton Star (Tropix) and incubated 40 minutes at 30° C. The β-Gal read-out (L) was measured using a Luminometer (Trilux, Wallach). The value of (OD₆₀₀ₙₘ×L) was calculated and interacting preys having the highest values were selected.

At this step of the protocol, diploid cell clones presenting interaction were isolated. The next step was now to identify polypeptides involved in the selected interactions.

### Example 3

Identification of Positive Clones

3.A. PCR on Yeast Colonies

Introduction

PCR amplification of fragments of plasmid DNA directly on yeast colonies is a quick and efficient procedure to identify sequences cloned into this plasmid. It is directly derived from a published protocol (Wang H. et al., Analytical Biochemistry, 237, 145-146, (1996)). However, it is not a standardized protocol and it varies from strain to strain and it is dependent of experimental conditions (number of cells, Taq polymerase source, etc). This protocol should be optimized to specific local conditions.

Materials

For 1 well, PCR mix composition was:

32.5 μl water,

5 μl 10×PCR buffer (Pharmacia),

1 μl DNTP 10 mM,

0.5 μl Taq polymerase (5u/μl) (Pharmacia),

0.5 μl oligonucleotide ABS1 10 Pmole/μl: 5′-GCGTTTGGAATCACTACAGG-3′, (SEQ ID No. 14)

0.5 μl oligonucleotide ABS2 10 pmole/μl: 5′-CACGATGCACGTTGAAGTG-3′. (SEQ ID No. 15)

1 N NaOH.

Experiment

The positive colonies were grown overnight at 30° C. on a 96 well cell culture cluster (Costar), containing 150 μl DO-Leu-Trp-His+ Tetracyclin with shaking. The culture was resuspended and 100 μl was transferred immediately on a Thermowell 96 (Costar) and centrifuged for 5 minutes at 4,000 rpm at room temperature. The supernatant was removed. 5 μl NaOH was added to each well and shaken for 1 minute.

The Thermowell was placed in the thermocycler (GeneAmp 9700, Perkin Elmer) for 5 minutes at 99.9° C. and then 10 minutes at 4° C. In each well, the PCR mix was added and shaken well.

The PCR program was set up as followed:

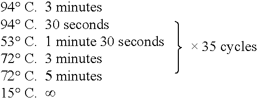

The quality, the quantity and the length of the PCR fragment was checked on an agarose gel. The length of the cloned fragment was the estimated length of the PCR fragment minus 300 base pairs that corresponded to the amplified flanking plasmid sequences.

3.B. Plasmids rescue from yeast by electroporation

Introduction

The previous protocol of PCR on yeast cell may not be successful, in such a case, plasmids from yeast by electroporation can be rescued. This experiment allows the recovery of prey plasmids from yeast cells by transformation of E. coli with a yeast cellular extract. The prey plasmid can then be amplified and the cloned fragment can be sequenced.

Materials

Plasmid Rescue

Glass beads 425-600 μm (Sigma)

Phenol/chloroform (1/1) premixed with isoamyl alcohol (Amresco)

Extraction buffer : 2% Triton X100, 1% SDS, 100 mM NaCl, 10 mM TrisHCl pH 8.0, 1 mM EDTA pH 8.0.

Mix ethanol/NH₄Ac: 6 volumes ethanol with 7.5 M NH₄ Acetate, 70% Ethanol and yeast cells in patches on plates.

Electroporation

SOC medium

M9 medium

Selective plates: M9-Leu+Ampicillin

2 mm electroporation cuvettes (Eurogentech)

Experiment

Plasmid Rescue

The cell patch on DO-Leu-Trp-His was prepared with the cell culture of section 2.C. The cell of each patch was scraped into an Eppendorf tube, 300 μl of glass beads was added in each tube, then, 200 μl extraction buffer and 200 μl phenol:chloroform:isoamyl alcohol (25:24:1) was added.

The tubes were centrifuged for 10 minutes at 15,000 rpm.

180 μl supernatant was transferred to a sterile Eppendorf tube and 500 μl each of ethanol/NH₄Ac was added and the tubes were vortexed. The tubes were centrifuged for 15 minutes at 15,000 rpm at 4° C. The pellet was washed with 200 μl 70% ethanol and the ethanol was removed and the pellet was dried. The pellet was resuspended in 10 μl water. Extracts were stored at −20° C.

Electroporation

Materials: Electrocompetent MC1066 cells prepared according to standard protocols (Sambrook et al. supra).

1 μl of yeast plasmid DNA-extract was added to a pre-chilled Eppendorf tube, and kept on ice.

1 μl plasmid yeast DNA-extract sample was mixed and 20 μl electrocompetent cells was added and transferred in a cold electroporation cuvette.

Set the Biorad electroporator on 200 ohms resistance, 25 μF capacity; 2.5 kV. Place the cuvette in the cuvette holder and electroporate.

1 ml of SOC was added into the cuvette and the cell-mix was transferred into a sterile Eppendorf tube. The cells were recovered for 30 minutes at 37° C., then spun down for 1 minute at 4,000×g and the supernatant was poured off. About 100 μl medium was kept and used to resuspend the cells and spread them on selective plates (e.g., M9-Leu plates). The plates were then incubated for 36 hours at 37° C.

One colony was grown and the plasmids were extracted. Check for the presence and size of the insert through enzymatic digestion and agarose gel electrophoresis. The insert was then sequenced.

### Example 4

Protein-Protein Interaction

For each bait, the previous protocol leads to the identification of prey polynucleotide sequences. Using a suitable software program (e.g., Blastwun, available on the Internet site of the University of Washington: http://bioweb.pasteur.fr/seganal/interfaces/blastwu.html) the identity of the mRNA transcript that is encoded by the prey fragment may be determined and whether the fusion protein encoded is in the same open reading frame of translation as the predicted protein or not.

Alternatively, prey nucleotide sequences can be compared with one another and those which share identity over a significant region (60 nt) can be grouped together to form a contiguous sequence (Contig) whose identity can be ascertained in the same manner as for individual prey fragments described above.

### Example 5

Making of Polyclonal and Monoclonal Antibodies

The protein-protein complex of columns 1 and 3 of Table 2 was injected into mice and polyclonal and monoclonal antibodies were made following the procedure set forth in Sambrook et al supra.

More specifically, mice were immunized with an immunogen comprising the above mentioned complexes or the epitopes described in Example 6 below conjugated to keyhole limpet hemocyanin using glutaraldehyde or EDC as is well known in the art. The complexes can also be stabilized by crosslinking as described in WO 00/37483. The immunogen was then mixed with an adjuvant. Each mouse receives four injections of 10 ug to 100 ug of immunogen, and after the fourth injection, blood samples were taken from the mice to determine if the serum contains antibodies to the immunogen. Serum titer is determined by ELISA or RIA. Mice with sera indicating the presence of antibody to the immunogen were selected for hybridoma production.

Spleens were removed from immune mice and single-cell suspension was prepared (Harlow et al 1988). Cell fusions are performed essentially as described by Kohler et al 1975). Briefly, P365.3 myeloma cells (ATTC Rockville, Md.) or NS-1 myeloma cells were fused with spleen cells using polyethylene glycol as described by Harlow et al (1989). Cells were plated at a density of 2×10⁵ cells/well in 96-well tissue culture plates. Individual wells are examined for growth and the supernatants of wells with growth are tested for the presence of complex-specific antibodies by ELISA or RIA using the protein-protein complex of columns 1 and 3 of Table 2 as a target protein. Cells in positive wells were expanded and subcloned to establish and confirm monoclonality.

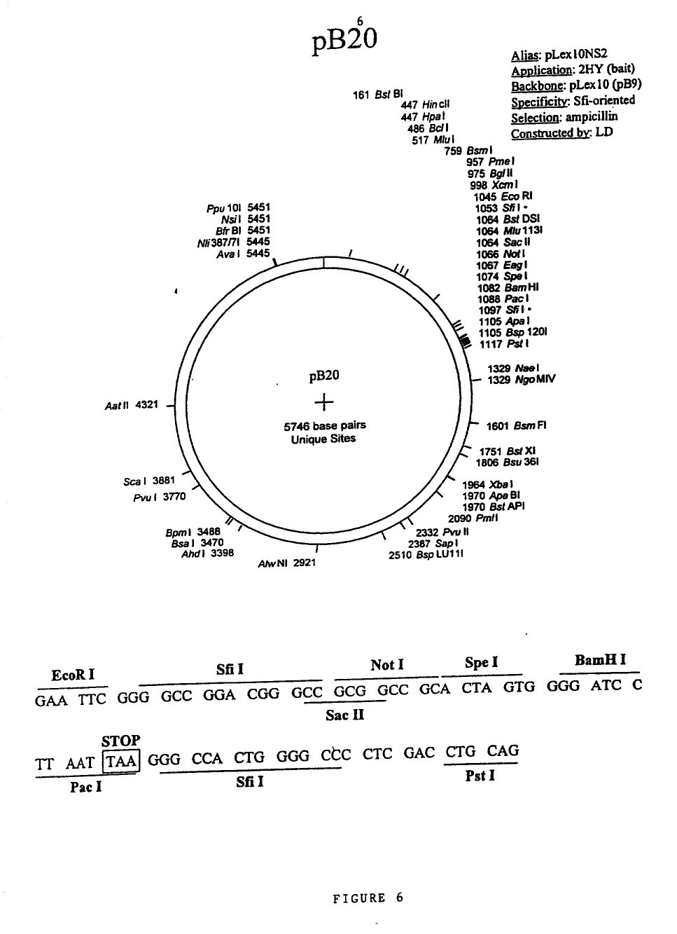

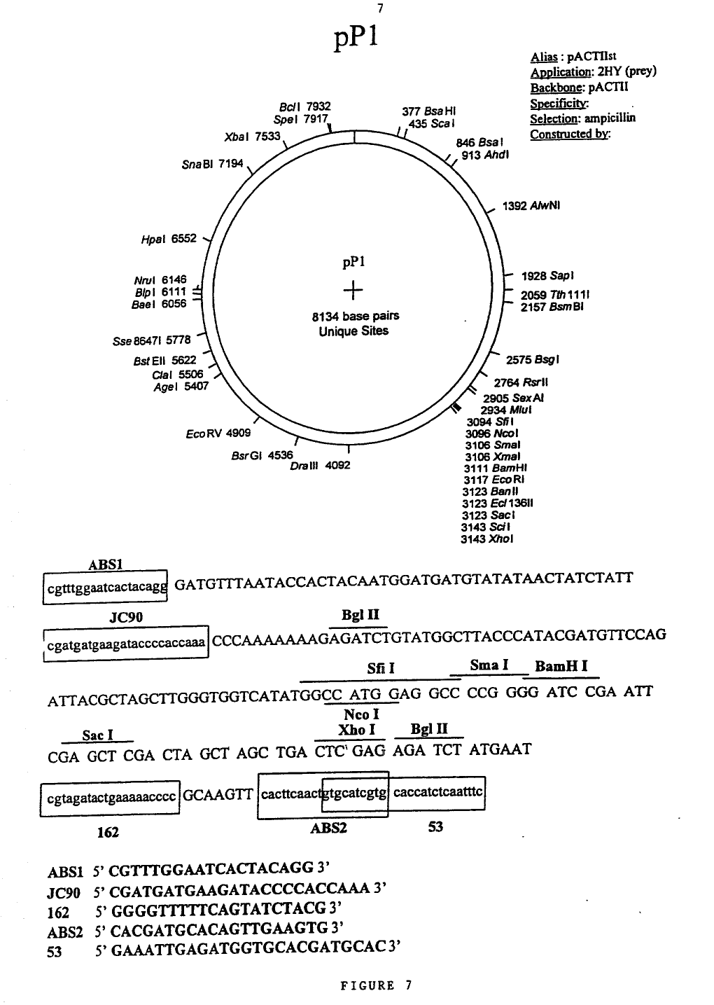

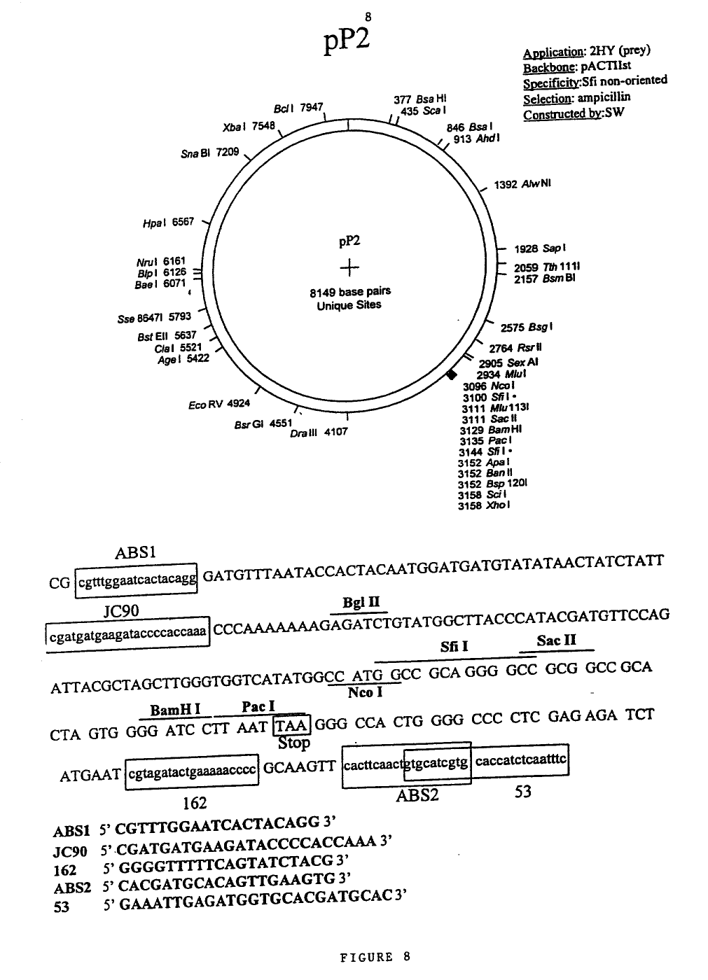

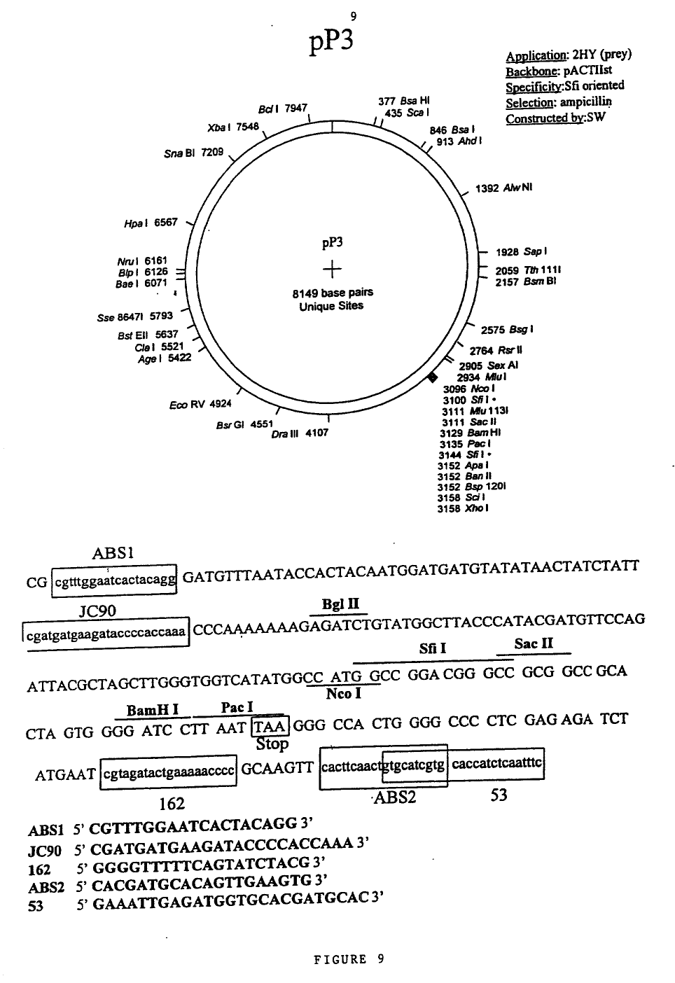

Clones with the desired specificities were expanded and grown as ascites in mice or in a hollow fiber system to produce sufficient quantities of antibodies for characterization and assay development. Antibodies were tested for binding to bait polypeptide of column 1 of Table 2 alone or to prey polypeptide of column 3 of Table 2 alone, to determine which are specific for the protein-protein complex of columns 1 and 3 of Table 2 as opposed to those that bind to the individual proteins or as described below in FIG. 6.

Monoclonal antibodies against each of the complexes set forth in columns 1 and 3 of Table 2 are prepared in a similar manner by mixing specified proteins together, immunizing an animal, fusing spleen cells with myeloma cells and isolating clones which produce antibodies specific for the protein complex, but not for individual proteins.

### Example 6

Further Antibody Production

The H3 antibody was generated against a bacterially expressed peptide of an epitope common to the three harmonin subclasses (PH3; amino acids 1 to 89). The H1b and H2b antibodies to harmonin b were generated against an epitope located in the PST domain of the protein (PHb:CRTGDPGHPADDWEA (SEQ ID No. 16); amino acids 636 to 649)

Three different rabbit polyclonal antibodies to human cadherin 23 were generated: cad-C was raised against a peptide in the cadherin 23 cytodomain (Pcad-C; ERNARTESAKSTPLHK (SEQ ID No. 17); amino acids 3,324to 3,339), cad-N was directed against two peptides in the extracellular region, namely Pcad-N1 (RGPRPLDRERNSSH (SEQ ID No. 18); amino acids 1,161 to 1,174) and Pcad-N2 (DIYYVLSSLDREKKDH (SEQ ID No. 19); amino acids 2,456 to 2,470) and cad-CN was from a rabbit immunized with all three peptides.

The specificity of the immunopurified antibodies was assayed by immunofluorescence and immunoblot analysis. Substitution of the preimmune sera for the purified anti-harmonin or anti-cadherin 23 antibody and preadsorption of the antibodies with the corresponding antigens, were used as negative controls.

### Example 7

Pull Down and in Vitro Binding Experiments

Transient transfections of the HEK293 cells were performed using PolyFect Transfection Reagent (Qiagen) following the manufacturer's instructions. Cells were collected 2 days after transfection and processed for pull down experiments as described in Kussel-Andermann et al., 2000, supra). The in vitro binding assays were performed using glutathione-sepharose (Amersham) or Tetra-link avidin resins (Promega) as described by Kussel-Andermann et al supra. Briefly, to test harmonin-myosin VIIa interaction, a bacterial lysate containing the GST-harmonin fusion protein was incubated with glutathione-resin (Amersham) for 90 minutes at 4° C. The resins were washed with binding buffer (Phosphate-buffered saline with 5% glycerol, 5 mM MgCl₂ and 0.1% Triton X-100)supplemented with a protease inhibitor cocktail (Roche) and then incubated with the his-tagged myosin VIIa or a his-tagged control protein, ezrin (amino acids 1 to 309) for 2 hours at 4° C. The resins were washed 4 times with binding buffer supplemented with 150 mM NaCl, and bound proteins were analysed by SDS-PAGE and immunoblotting, using the ECL chemiluminescence system (Amersham).

### Example 8

Immunofluorescence and Electron Microscopy Analysis

HeLa cell lines were cultivated in 10% FCS-supplemented DMEM. Transient transfections of these cells were performed using Effectene (Qiagen) following the manufacturer's instructions. Immunohistofluorescence analysis was carried out on fixed cells and cryostat sections of inner ears, as previously described by Kussel-Andermann et al, supra). Cells and tissue sections were analysed with a laser scanning confocal microscope (LSM-540, Zeiss). For immunoelectron microscopy, cochleas from P20 mice (CD1 strain) were labelled with affinity purified cad-N anti-cadherin 23 antibodies or, as a control, nonimmune rabbit IgG, as essentially described by Goodyear and Richardson, J. Neurosci. 19 pgs. 3761-72 (1999)).

The following mouse monoclonal antibodies were used: anti-Myc (clone 9E10, Santa Cruz); anti-His (Santa Cruz), anti-GST (Amersham);anti-vinculin (Sigma). For myosin VIIa detection in Western blots and harmonin-myosin VIIa double labelling experiments, a monoclonal mouse antibody (Farida-Nato, IP) raised against a human myosin VIIa tail fragment (amino acids 905 to 1,032, Genebank Accession No. U39226) was used. Several polyclonal rabbit antibodies were used, which were directed against myosin VIIa (El-Amraoui et al Hum. Mol Genet 5, 1171-8 (1996)), harmonin (Kobayashi et al., Gastroenterology 117, 823 -30 (1999)), espin (Zheng et al., Cell 102 377-85 (2000)) and stereocilin (Verpy et al., Nat Genet 29, 345-9 (2001)). Secondary antibodies coupled with Cy-2 or Cy-3 were from Amersham (Molecular Probes).

### Example 9

BAPTA and Subtilisin Treatment of Mouse Cochlear Cultures

Organotypic cochlear cultures were prepared from P2 mice (CD1 strain) essentially as previously described by Russell and Richardson, Hear Res 31, 9-24 (1987)). After 1 day in vitro, cultures were washed twice briefly with 10 mM Hepes (pH 7.2)buffered with Hank's balanced salt solution (HBHBSS). The cultures were then incubated for 15 minutes at room temperature in either a control medium (HBHBSS), Ca²⁺-free HBHBSS containing 5 mM BAPTA (Sigma) or HBHBSS containing 50 μg/ml subtilisin (Protease Type XXIV; Sigma). In one set of experiments subtilisin treatment was done in the presence of 5 mM Ca²⁺ with appropriate high Ca²⁺-HBHBSS controls run in parallel. Following treatment, cultures were washed once briefly in HBHBSS and fixed in 3.7% formaldehyde/0.025% glutaraldehyde buffered with 0.1 M sodium phosphate pH 7.4 for one hour, washed three tines with phosphate buffered saline, preincubated for 1 hour in Tris-buffered (10 mM, pH 7.4) saline containing 10% horse serum and incubated overnight with the cad-N anti-cadherin 23 antibody (1 g/ml). Preimmune serum or rabbit serum were used as controls.

### Example 10

Actin Bundling and Cosedimentation Assays

50 μm of G-actin (Molecular Probes) was polymerised by incubation for 30′ at 37° C. in a high salt buffer containing 50 mM KCL and 2 mM MgCl₂. Indicated amounts of GST-harmonin b or GST-CC2-Cter truncated form were incubated 30 minutes with 10 μM of F-actin reconstituted from actin powder (Pardee and Spudich, J Cell Biol 93, 648-54, (1982) at 37° C. Actin polymers were then observed in the fluorescence microscope labelled with Rhodamin-phallodin or were then analysed by electron microscopy after negative staining according to Harris, J. Electron Microsc. Tech 18, 269-76 (1991)). Cosedimentation assays were performed by mixing GST-harmonin with F-actin followed by centrifugation (30 minutes at 18,000 g). The comparable amount of supernatant and pellet fractions were subjected to SDS-PAGE and analysed with the H1b anti-harmonin antibody and Coomassie blue staining.

### Example 11

Modulating Compounds Identification

Each specific protein-protein complex of columns 1 and 3 of Table 2 may be used to screen for modulating compounds.

One appropriate construction for this modulating compound screening may be:

bait polynucleotide inserted in pB6 or pB27;

prey polynucleotide inserted in pP6;

transformation of these two vectors in a permeable yeast cell;

growth of the transformed yeast cell on a medium containing compound to be tested,

and observation of the growth of the yeast cells.

Results

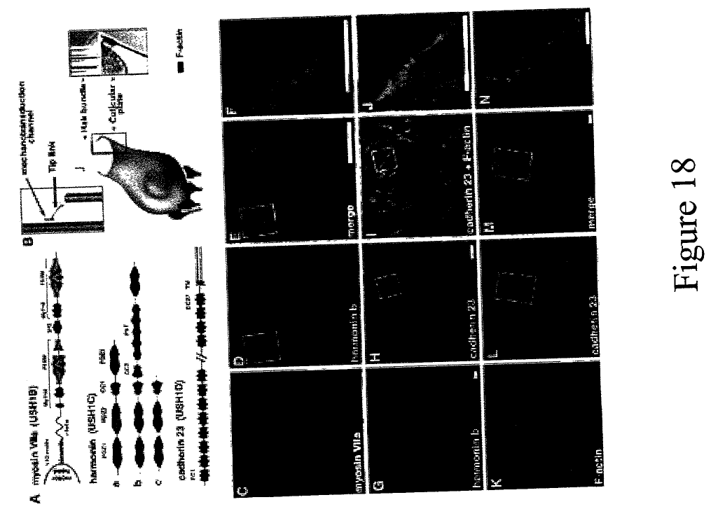

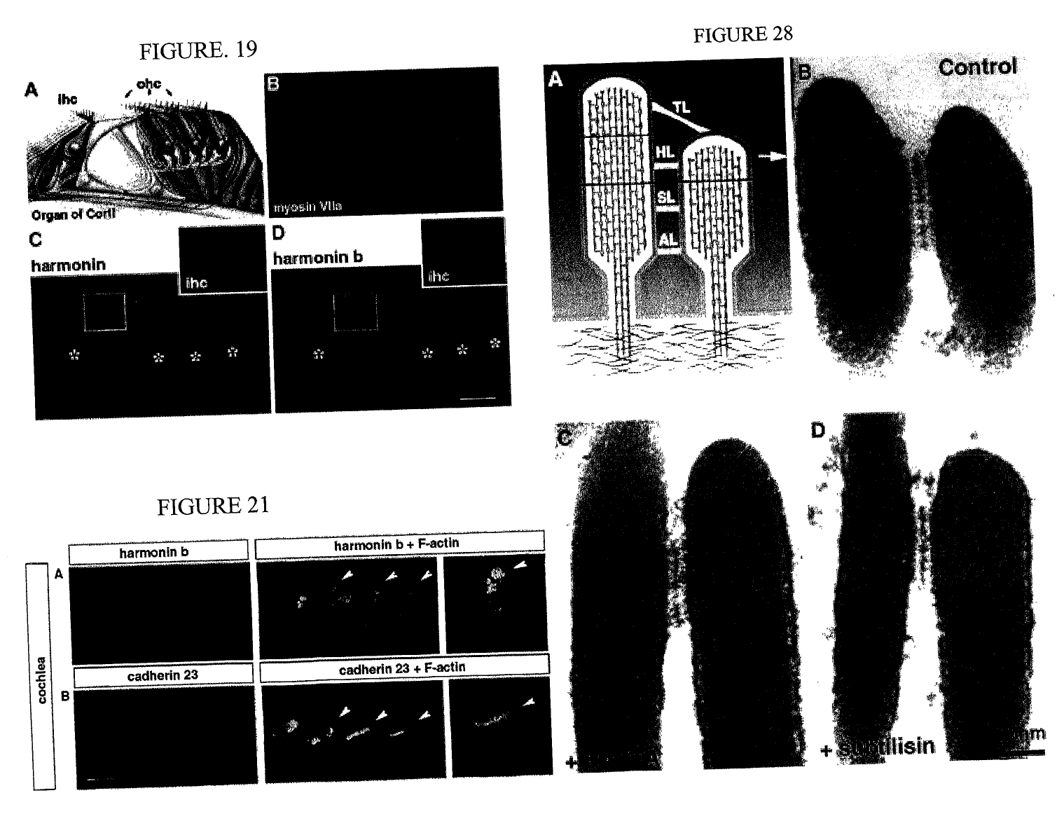

The distribution of myosin VIIa, harmonin and cadherin 23 in early postnatal PO-P30 inner ear of both the mouse and rat was analyzed by immunofluorescence microscopy. In agreement with previous studies of El-Amraoui et al., Hum Mol Genet 5, 1171-8 (1996) and Hasson et al PNAS USA 92, 9815-9 (1995), myosin VIIa was observed in the hair bundle and throughout the body of the hair (See, FIG. 18, C,E,F and FIG. 19) Harmonin, was detected with an antibody that recognizes an epitope common to all three classes, which was found in both the hair bundle and the underlying cuticular plate (FIG. 18B).

Since a previous analysis of harmonin transcripts had indicated that the class b isoforms are largely restricted to the inner ear (Verpy et al. Nat Genet 26, 51-5, (2000), two antibodies were raised against the PST domain that is present only in harmonin b (see above Examples and FIG. 18A) and explored the distribution of this subclass. With both antibodies harmonin was detected in the hair bundle where it formed punctate spots located at the distal ends of many of the stereocilia (FIGS. 18D-G). Harmonin B was not found in the cuticular plate (FIGS. 18D-G and FIG. 19D). Three rabbit immune sera were raised against the extracellular and intracellular regions of cadherin 23 (see examples above). These antibodies revealed the presence of cadherin 23 in the hair bundle, where it was concentrated at the apex of stereocilia (FIGS. 8H-J,L-N).

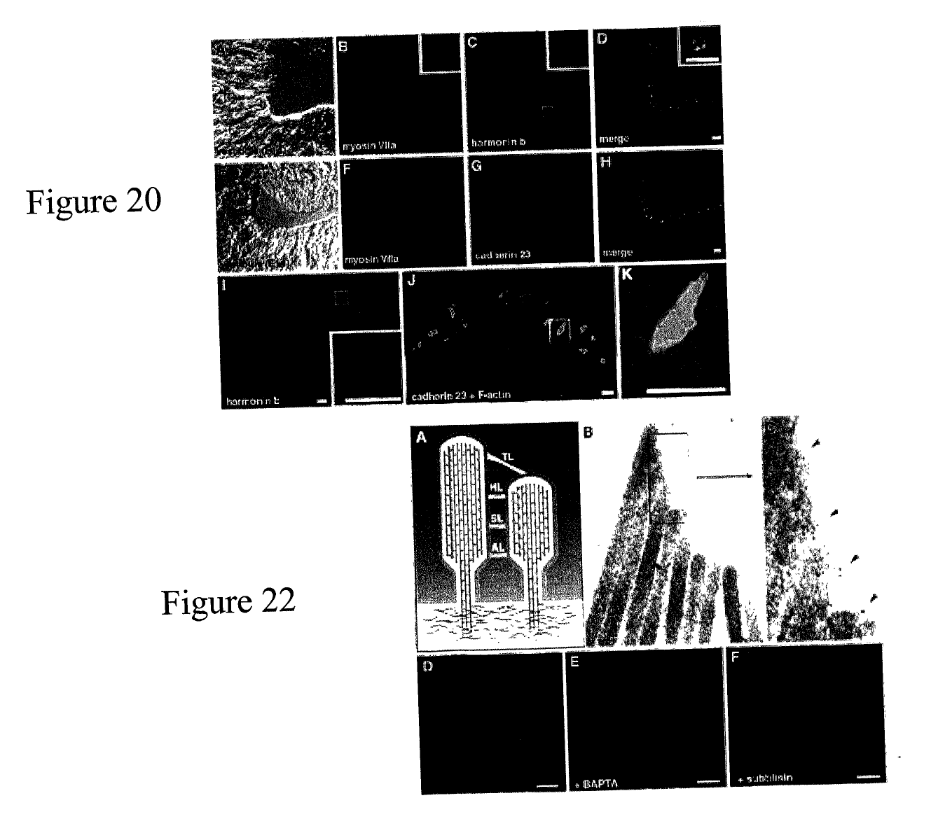

The spatio-temporal distribution of harmonin and cadherin 23 during the initial stages of hair-bundle formation and differentiation to determine whether they play a role in these processes was undertaken. In the mouse, stereocilia sprout from the apical of vestibular and cochlear hair cells at E13 and E15, respectively (Denman-Johnson and Forge J. Neurocytol. 28, 821-35 (1999); Nishida et al, J. Comp. Neor ; 395, 18-28 (1998). In the cochlea, the differentiation of hair cells proceeds from the base to the apex of the organ of Corti and by P4-P6, the hair bundles attain their final shape (Nishida et al, supra 1998). In the mouse vestibule, double immunolabels for harmonin b and myosin VIIa, or cadherin 23 and myosin VIIa showed that the 3 proteins co-localized in the stereocilia as soon as they emerge from the apical pole of hair cells; i.e., from E12 onwards (FIGS. 20A-H). Likewise myosin VIIa, cadherin 23 and harmonin b were first detected in hair bundles in the base of the mouse cochlea at E15. By E17, hair cells throughout the length of the cochlea expressed all three proteins (data not shown). Detailed analysis of the hair bundles by confocal microscopy showed harmonin b (FIG. 20I) and cadherin 23 (FIGS. 20J,K) were distributed along the entire length of the emerging hair bundles. However, from E16 in the vestibule and from P0 in the cochlea, harmonin b and cadherin 23 became progressively restricted to the distal part of the elongating stereocilia in both vestibular (FIGS. 18D-F,H-J.) and cochlear hair cells (FIGS. 21A-B). By P30 in the vestibule, harmonin b (FIG. 18G) and cadherin 23 (FIGS. 18L-N) could only be detected in a proportion of the hair bundles. In the cochlea, neither protein could be detected in the hair bundle after this period.

Although the postnatal loss of immuno-detectable cadherin 23 from hair bundles in the cochlea suggests it is unlikely to be a component of any of the link types (see, FIG. 22A) known to be associated with the surface of the hair bundle, tip links have been shown to develop from an extensive array of apically-located, lateral links that are found around the tips of stereocilia in immature hair bundles (Pickles et al., Hear Res 15, 103-121991).

Furthermore, by immunoelectron microscopy extracellular cadherin 23 epitopes (see Examples) were detected between adjacent stereocilia, with an especially high density found at the tips of the stereocilia (FIG. 22B,C) on immature hair bundles. Cadherin 23 was then tested to determine whether its properties are consistent with it being a component of tip links. Mouse cochlear cultures prepared at P2 were labeled with an antibody to cadherin 23 after treatment with either the calcium chelator (BAPTA, 5 mM) or the serine protease subtilisin. (FIGS. 22D-F). In whole mount preparations of control cultures, the cadherin 23 labeling revealed a highly organized shape of hair bundles on the inner and outer hair cells (FIG. 22D). In all, BAPTA- treated cultures (n=14), the staining was unaffected (FIG. 22D). In contrast, in all subtilisin-treated cultures (n=12) the cadherin 23 labeling of hair bundles was no longer observed, irrespective of the extracellular calcium concentration used during enzyme treatment (FIG. 22F). Thus, BAPTA sensitive but subtilisin resistant, tip links as well as the BAPTA/subtilisin insensitive horizontal top links (See, Goodyear & Richardson Hear Res 15, 103-12 1999) for the chick; and FIGS. 28A-D in the mouse) are unlikely to be composed of cadherin 23.

These results show that myosin VI a, harmonin b and cadherin 23 are present in the hair bundles from the earliest stage of differentiation. Even though the three molecules are found throughout emerging stereocilia, harmonin b and cadherin 23 rapidly become restricted to the distal end of the hair bundle and may only be expressed at high levels transiently during development, whereas myosin VIIa remains distributed along the structure during the entire lifetime of the hair cell.

Harmonin b is an F-actin Bundling Protein

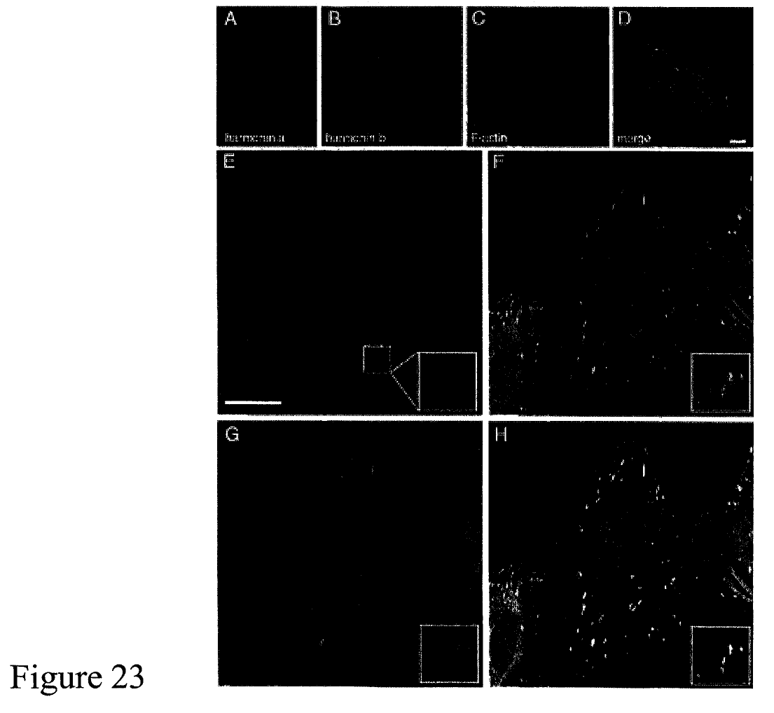

To address the function of harmonin, a representative of each of the three isoform classes was transfected into HeLa cells. In cells producing harmonin a or harmonin c, irrespective of the level of protein expression, the protein was uniformly distributed throughout the cell body (FIG. 23A). In contrast cells expressing harmonin b, the protein was associated with filamentous structure (FIG. 23B). Double immunolabeling with antibodies to α tubulin, β-tubulin, cytokeratin 18, vimentin, or a pan cytokeratin antibody showed that harmonin b was not associated with either microtubules or intermediate filaments. In contrast, harmonin b co-localized with actin filaments that were labeled with TRITC-phalloidin (FIGS. 23B-D). To determine which domain(s) may target harmonin b to the actin cytoskeleton, a series of truncated mutants were expressed in HeLa cells. The shortest construct of harmonin b used that co-localized with actin filaments encompasses the second-coiled coil domain through to the C-terminal end (CC2-Cter, amino acids 405-859). The pattern of full length harmonin b staining in transfected HeLa cells varied with the expression level. In cells producing low levels of harmonin b, the protein was restricted to and highly concentrated at the tips (barbed ends) of actin stress fibers, where these fibers are anchored to substrate adhesion sites (FIGS. 23E-H). Vinculin, a major component of the focal adhesion plaques (Zamir and Geiger, J Cell Sci 114, 3577-9 2001), co-localized with harmonin b but the labeling of the latter overlapped only with the proximal part of the vinculin staining (FIG. 23G). In cells producing high levels of harmonin b, the actin cytoskeleton was disrupted and long (several μm), curvy filament bundles containing harmonin b and actin, were observed scattered throughout the cell (FIG. 23B). The dynamics of GFP-harmonin b distribution in living HeLa cells was studied by digital fluorescence microscopy and revealed that the stress fibers that bind harmonin b and are rooted to focal adhesion sites eventually transform into the long and curvy filament bundles and maintain anchorage in the focal adhesion plaques, To further characterize the behavior of the actin filaments in the presence of harmonin b, cells were treated with either latrunculin A, which binds to and sequesters actin monomers or cytochalsin D that binds to the barbed end of actin filaments and alters polymerization. With either of these drugs, both the cortical actin filaments and the harmonin b-unlabeled stress fibers were disrupted, whereas the harmonin b-associated actin stress fibers were unaffected (FIGS. 24A-D).

These morphological studies indicate that transfected harmonin b is associated with the actin cytoskeleton. To test whether harmonin b binds directly to actin filaments, in vitro binding assays were performed with expressed harmonin b and purified F-actin. Harmonin b and rhodamine-phalloidin labeled actin filaments were incubated together and then samples were visualized by light microscopy or negatively stained for electron microscopy. Actin filaments were collected into large bundles in the presence of harmonin b as shown in FIGS. 24E,G. To determine whether the harmonin b was associated with these actin bundles, harmonin b and actin filaments were mixed to allow bundling and then the bundles were separated from soluble proteins and single actin filaments by low-speed centrifugation. The pelleting assays were done in the presence of either GST alone, full length GST-tagged harmonin b or a GST-tagged harmonin CC2Cter fragment. With GST alone, almost all of the rabbit skeletal muscle F-actin remained in the supernatant fraction. In contrast, in the presence of the GST-tagged harmonin b or the CC2-Cter fragments, the vast majority of F-actin was recovered in the pellet along with harmonin (FIG. 24H). This actin-bundling activity of harmonin b was unaffected by high calcium concentration (10 mM) or calcium chelating agents (10 mM EGTA) (data not shown).

Harmonin Binds to Cadherin 23

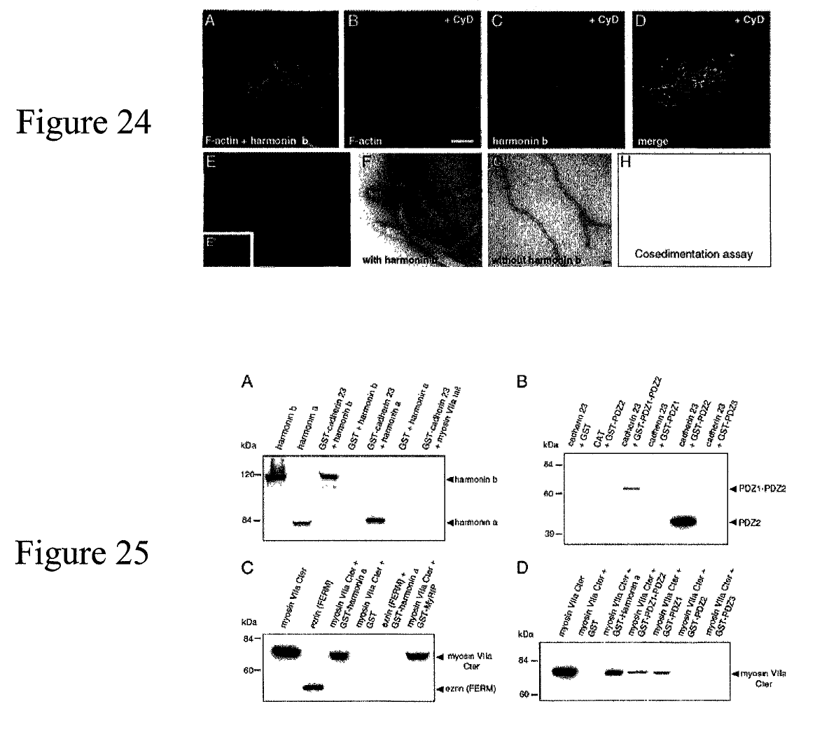

The colocalization of harmonin b and cadherin 23 in the distal part of the maturing stereocilia and in co-transfected HeLa cells (see, FIG. 25A) indicated that these molecules may physically interact. Therefore, whether an interaction could be detected by Pull down assays was performed. Extracts of transfected HEK293 cells producing either harmonin b or the myosin VIIa tail (amino acids 848-2215) were incubated with immobilized GST-tagged cadherin 23 cytodomain (amino acids 3086-3354) or GST alone. Significant recovery of harmonin b was obtained with the GST-tagged cadherin 23 cytodomain, but not with GST alone. In contrast, the myosin VIIa tail was not recovered (FIG. 25A). The interaction between cadherin 23 and harmonin was further analyzed by an in vitro binding assay (FIG. 25B). Different harmonin fragments were produced (see Examples) and incubated with immobilized biotin-tagged cadherin 23 cytodomain. Both the PDZ1-PDZ2 peptide (amino acids 138-403) and the PDZ2 domain alone (amino acids 189-307) of harmonin (FIG. 25B) bound to the cadherin 23 cytodomain, whereas binding was not observed with either PDZ1 or PDZ3 (FIG. 25B).

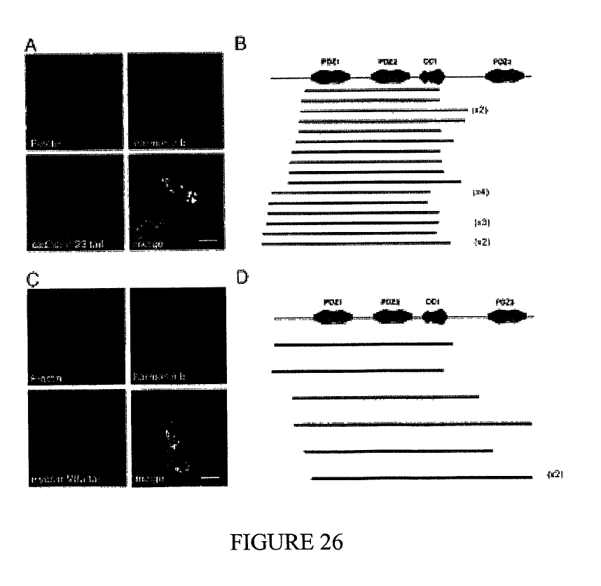

These findings were substantiated by the results of a yeast two-hybrid screen for ligands of cadherin 23. 16 independent clones encoding harmonin were isolated from the inner ear sensory epithelium cDNA library using the last 268 amino acids (amino acids 3086-3354) of the cadherin 23 cytoplasmic domain as the bait. The overlapping sequences of these clones encode the PDZ1 and PDZ2 domains (amino acids 114-322) of harmonin (see FIG. 26b). These results, in conjunction with the pull down assays, indicate that the PDZ2 domain of harmonin binds to cadherin 23.

Myosin VIIa Transports Harmonin b in the Stereocilia

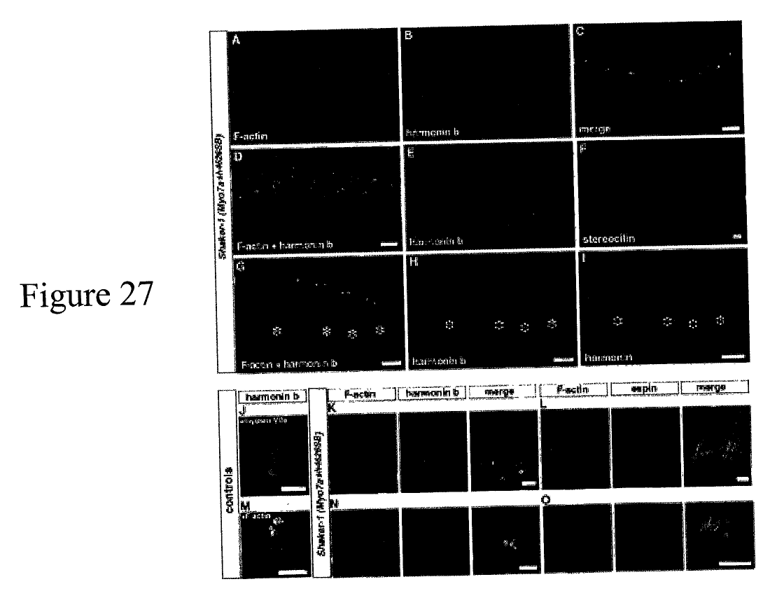

Myosin VIIa has been recently shown to be a bona fide motor that moves along actin filaments (Udovichenko et al., J Cell Sci 115, 445-50 2002). Although the directionality of myosin VIIa movement along actin filaments has not been determined, except for myosin IV, all myosins tested to date are actin plus-end directed. Thus it is expected that myosin VIIa translocates towards the plus-ends of actin filaments near the stereocilium tip and thus may tow molecules to this location. To address this possibility, the distribution of cadherin 23 and harmonin b in the Myo7a^{4626SB} shaker-1 mice was studied. These mice carry a premature stop codon in the motor domain of myosin VIIa, and have severely disorganized hair bundles (Mburu et al., 1997). In both the vestibular (FIGS. 27A-E,K) and cochlear (FIGS. 27G,H,N) hair cells of Myo7a^{4626SB} shaker-1 mice, and at all stages examined, namely E20, P0, P2, P4, P8, P15 and P30, no detection of harmonin b in the stereocilia was found. Instead, harmonin b was found to be organized in a circle of bead-like foci located around the periphery of the cuticular plate (FIGS. 27B-E,G,H,K). In contrast, harmonin a/c (FIG. 27I) and cadherin 23 (not shown) were both present and distributed as in wild type mice in the stereocilia of Myo7a^{4626SB} mutant mice. Likewise, two other proteins of the stereocilia, namely stereocilin, a protein of as yet unknown function (Verpy et al. Nat Genet 29, 345-9, 2001), and espin, an actin cross-linking protein (Zheng et al., Cell 102, 377-85 2000), also had the same distribution along the length of the stereocilium in control and mutant mice (see FIGS. 27F,L,O). This strongly suggests that myosin VIIa is involved in the transport of harmonin b toward the tip region of the stereocilium.

Such a proposal implies that the myosin VIIa tail and harmonin b physically interact. Consistently, in cotransfected HeLa cells producing harmonin b and either the entire myosin VIIa tail or its C-terminal MyTH4+FERM repeat (amino acids 1750-2215), the myosin VIIa fragments entirely co-localized with harmonin b and actin (see, FIG. 26C) Moreover, the presence of the myosin VIIa fragments profoundly modified the harmonin b-actin pattern. The long curvy filament bundles that were observed in the absence of myosin VIIa tail changed into large puncta. Similar co-transfection experiments with harmonin a or c argued in favor of an interaction of the three harmonin subclasses with myosin VIIa (not shown). We thus tested the direct binding of harmonin a to myosin VIIa by in vitro binding assays. The C-terminal MyTH4+FERM repeat of myosin VIIa did interact with GST-tagged harmonin a, as did GST-MyRIP (myosin VIIa and rab interacting protein; (El-Amraoui et al. EMBO Rep 3, 463-70, 2002)) fragment (used as a positive control), whereas both failed to bind to GST alone (see FIG. 25C). In contrast, the ezrin FERM domain did not bind to GST-harmonin a (FIG. 25C). By using various constructs expressing harmonin PDZ domains, it was shown that only PDZ1 has affinity for myosin VIIa (FIG. 25D).

A yeast two-hybrid screen corroborated these results. Using a C-terminal fragment of the myosin VIIa tail containing the SH3, MyTH4 and FERM domains (amino acids 1605-2215) as the bait, six independent clones encoding harmonin were isolated from the inner ear two-hybrid cDNA library. Their overlapping sequences encode a harmonin fragment (amino acids 90-368)containing PDZ1, PDZ2 and part of the CC1 domain (see, FIG. 26D).

Together these results suggest that the PDZ1 domain of harmonin binds to myosin VIIa.

In conclusion, the above results show all three are components of the mechanosensory hair bundle from (the onset) of its emergence. Harmonin b is shown to be an F-actin bundling protein that binds to the cytoplasmic domain of cadherin 23, thereby anchoring this adhesion molecule of the hair-bundles's surface to the actin-rich cores of its stereocilia. Moreover, harmonin b is absent from the disorganized hair bundles of myosin VIIa mutant mice, and interacts directly with myosin VIIa, suggesting myosin VIIa conveys harmonin b to the hair bundle. Thus it can be concluded that an early inter-stereocilial adhesion process is required to shape a coherent hair bundle. It relies on myosin VIIa, harmonin b and cadherin 23 acting together as a functional network, and is disrupted in USH1B, USH1C and USH1D.

The following results obtained from these Examples, as well as the teachings in the specification are set forth in the Tables below.

All non-patented websites are incorporated herein by reference.

While the invention has been described in terms of the various preferred embodiments, the skilled artisan will appreciate that various modifications, substitutions, omissions and changes may be made without departing from the scope thereof. Accordingly, it is intended that the present invention be limited by the scope of the following claims, including equivalents thereof.

[image]

[image]

[image]

TABLE 2
bait-prey interactions

| | 2. Bait | |
|---|---|---|
| | Sequence | |
| | ID (nucleic | |
| | acid/ | |
| 1. Bait Name | amino acid) | 3. Prey Name |
| Human Myosin VIIA | 1 | ref\|NM_0236491.1\| Mus musculus |
| | | RIKEN cDNA 2010016F01 gene |
| | | (2010016F01Rik), mRNA |
| | | (hgx114v2 harmonin) |
| Human Cadherin 23 | 2 | hgx110 (Mouse Harmonin isoform |
| isoform_v1 | | a1 (Ush1c)-alternatively |
| | | spliced form of hgx125 prey16908) |
| | | m2010016F01Rik mUsh1c |

TABLE 3

SID ®

| | 2: Bait | | | | | |
|---|---|---|---|---|---|---|
| | nucleic | | 4:SID | | 6: SID | |
| | acid | 3: nucleic | | amino-acid | | |
| 1: Bait | SEQ | Prey | acid ID | | ID | |
| name | ID No. | name | No. | 5: SID nucleic acid sequence | No. | 7: SID amino-acid sequence |
| Human | 1 | hgx11 | 5 | TTGGACCGTCTGCACCCAGAAGGTCTCGGCCTCA | 7 | LDRLHPEGLGLSVRGGLEF |
| Myosin | | 4v2 | | GCGTGCGTGGAGGCCTGGAATTTGGCTGTGGACT | | GCGLFISHLIKGGQADSVGL |
| VIIA | | | | CTTTATCTCCCACCTCATCAAAGGTGGCCAGGCAG | | QVGDEIVRINGYSISSCTHEE |
| | | | | ACAGCGTTGGGCTTCAGGTAGGGGATGAAATTGT | | VINLIRTKKTVSIKVRHIGLIPV |
| | | | | CCGGATCAACGGCTATTCCATCTCTTCCTGTACCC | | KSSPEESLKWQYVDQFVSE |
| | | | | ATGAGGAAGTCATCAACCTGATCCGCACCAAGAAG | | SGGVRGGLGSPGNRTTKEK |
| | | | | ACCGTGTCCATCAAAGTGAGACACATCGGACTGAT | | KVFISLVGSRGLGCSISSGPI |
| | | | | CCCTGTGAAGAGCTCTCCTGAGGAGTCCCTCAAAT | | QKPGIFVSHVKPGSLSAEVG |
| | | | | GGCAGTATGTGGATCAGTTCGTGTCGGAATCTGG | | LETGDQIVEVNGIDFTNLDH |
| | | | | GGGTGTGCGAGGTGGCTTGGGCTCACCTGGCAAT | | KEAVNVLKSSRSLTISIVAGA |
| | | | | CGGACAACCAAGGAGAAGAAGGTGTTTATCAGTCT | | GRELFMTDRERLEEARQRE |
| | | | | AGTGGGCTCTCGGGGCCTGGGCTGCAGCATCTCC | | LQRQELLMQKRLAMESNKIL |
| | | | | AGTGGCCCCATCCAGAAGCCTGGCATCTTCGTCA | | QEQQEMERQRRKEIAQKAA |
| | | | | GCCACGTGAAGCCTGGCTCCCTGTCTGCAGAGGT | | EENERYRKEMEQISEEEE |
| | | | | GGGGTTAGAGACAGGAGACCAGATTGTGGAAGTC | | |
| | | | | AATGGCATAGACTTCACCAACCTGGACCACAAGGA | | |
| | | | | GGCTGTGAATGTCCTGAAGAGCAGCCGCAGCCTG | | |
| | | | | ACCATCTCCATCGTTGCTGGAGCCGGCCGGGAGC | | |
| | | | | TGTTCATGACGGACCGGGAACGGCTGGAGGAGGC | | |
| | | | | ACGGCAGCGTGAGCTGCAACGGCAGGAACTCCTC | | |
| | | | | ATGCAGAAGCGGCTGGCCATGGAGTCCAACAAGA | | |
| | | | | TCCTCCAGGAGCAGCAGGAGATGGAGCGCCAGAG | | |
| | | | | GAGAAAGGAGATCGCCCAGAAGGCTGCCGAGGA | | |
| | | | | GAATGAGAGATACCGGAAGGAGATGGAACAGATC | | |
| | | | | TCGGAGGAGGAAGAG | | |
| Human | 2 | hgx11 | 6 | GGCAGACAGCGTTGGGCTTCAGGTAGGGGATGAA | 8 | ADSVGLQVGDEIVRINGYSIS |
| Cadherin | | 0 | | ATTGTCCGGATCAACGGCTATTCCATCTCTTCCTG | | SCTHEEVINLIRTKKTVSIKV |
| 23 | | | | TACCCATGAGGAAGTCATCAACCTGATCCGCACCA | | RHIGLIPVKSSPEESLKWQY |
| isoform_v | | | | AGAAGACCGTGTCCATCAAAGTGAGACACATCGG | | VDQFVSESGGVRGGLGSPG |
| 1 | | | | ACTGATCCCTGTGAAGAGCTCTCCTGAGGAGTCC | | NRTTKEKKVFISLVGSRGLG |
| | | | | CTCAAATGGCAGTATGTGGATCAGTTCGTGTCGGA | | CSISSGPIQKPGIFVSHVKPG |
| | | | | ATCTGGGGGTGTGCGAGGTGGCTTGGGCTCACCT | | SLSAEVGLET |
| | | | | GGCAATCGGACAACCAAGGAGAAGAAGGTGTTTA | | |
| | | | | TCAGTCTAGTGGGCTCTCGGGGCCTGGGCTGCAG | | |
| | | | | CATCTCCAGTGGCCCCATCCAGAAGCCTGGCATC | | |
| | | | | TTCGTCAGCCACGTGAAGCCTGGCTCCCTGTCTG | | |
| | | | | CAGAGGTGGGGTTAGAGACAGG | | |

What is claimed is:

1. A complex of protein-protein interactions as defined in columns 1 and 3 of Table 2.

2. A complex of polynucleotides as defined in SEQ ID Nos. 1 or 2 encoding for the polypeptides.

3. A recombinant host cell expressing the interacting polypeptides as defined in Table 1.

4. A method for selecting a modulating compound comprising:
(a) cultivating a recombinant host cell with a modulating compound on a selective medium and a reporter gene the expression of which is toxic for said recombinant host cell wherein said recombinant host cell is transformed with two vectors:
(i) wherein said first vector comprises a polynucleotide in column 1 of Table 2 encoding a first hybrid polypeptide and a DNA bonding domain;
(ii) wherein said second vector comprises a polynucleotide in column 3 of Table 2 encoding a second hybrid polypeptide and an activating domain that activates said toxic reporter gene when the first and second hybrid polypeptides interact;
(b) selecting said modulating compound which inhibits the growth of said recombinant host cell.

5. A modulating compound obtained by the method of claim 4.

6. A vector comprising the polynucleotide comprising the SEQ ID Nos. 1 or 2.

7. A fragment of a polypeptide comprising SEQ ID Nos. 3 or 4.

8. A variant of a polypeptide comprising SEQ ID Nos. 3 or 4.

9. A recombinant host cell containing the vectors according to claim 6.

10. A pharmaceutical composition comprising a modulating compound of claim 5 and a pharmaceutically acceptable vehicle.

11. A pharmaceutical composition comprising the recombinant host cells of claim 9 and a pharmaceutically acceptable vehicle.

12. A protein chip comprising the polypetides of claims 7 or 8.

13. A polynucleotide comprising SEQ ID Nos. 5 and 6 or a fragment or variant thereof.

14. A polypeptide of SEQ ID Nos. 7 and 8 or a fragment or variant thereof.

15. A method to detect Usher type I syndrome said method comprising:
obtaining a biological sample from a subject; and
identifying a defect in the proteins which are myosin VIIa, harmonin b and cadherin 23.


SEQUENCE LISTING (8 sequences)

SEQ ID NO: 1 - DNA, 1024 bp, Homo sapiens
tctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctct

SEQ ID NO: 2 - DNA, 784 bp, Homo sapiens
atggccggacgggccgcggtcatgaactggtactacaggactgtacacaagaggaagctcaaggccattgtggctggctcagctgggaatcgtggcttcatcgacatcatggacatgcctaacaccaacaagtactcctttgatggagccaaccctgtgtggctggatcccttctgtcggaacctggagctggccgcccaggcggagcatgaggatgacctaccggagaacctgagtgagatcgccgacctgtggaacagccccacgcgcacccatggaacttttgggcgtgagccagcagctgtcaagcctgatgatgaccgatacctgcgggctgccatccaggagtatgacaacattgccaagctgggccagatcattcgtgaggggccaatcaagctgatacagactgagctggacgaggagccaggagaccacagcccagggcagggtagcctgcgcttccgccacaagccaccagtggagctcaaggggcccgatgggatccatgtggtgcacggcagcacgggcacgctgctggccaccgacctcaacagcctgcccgaggaagaccagaagggcctgggccgctcgctggagacgctgaccgctgccgaggccactgccttcgagcgcaacgcccgcacagaatccgccaaatccacacccctgcacaaacttcgcgacgtgatcatggagacccccctggagatcacagagctgtgactagacagggaagccttgtgggtgtgagcagcacccactagtggggatccttaattaa

SEQ ID NO: 3 - protein, 611 aa, Homo sapiens
  Translation of SEQ ID No1
SKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 4 - protein, 241 aa, Human
  Translation of SEQ ID No2
MAGRAAVMNWYYRTVHKRKLKAIVAGSAGNRGFIDIMDMPNTNKYSFDGANPVWLDPFCRNLELAAQAEHEDDLPENLSEIADLWNSPTRTHGTFGREPAAVKPDDDRYLRAAIQEYDNIAKLGQIIREGPIKLIQTELDEEPGDHSPGQGSLRFRHKPPVELKGPDGIHVVHGSTGTLLATDLNSLPEEDQKGLGRSLETLTAAEATAFERNARTESAKSTPLHKLRDVIMETPLEITEL

SEQ ID NO: 5 - DNA, 837 bp, Human
ttggaccgtctgcacccagaaggtctcggcctcagcgtgcgtggaggcctggaatttggctgtggactctttatctcccacctcatcaaaggtggccaggcagacagcgttgggcttcaggtaggggatgaaattgtccggatcaacggctattccatctcttcctgtacccatgaggaagtcatcaacctgatccgcaccaagaagaccgtgtccatcaaagtgagacacatcggactgatccctgtgaagagctctcctgaggagtccctcaaatggcagtatgtggatcagttcgtgtcggaatctgggggtgtgcgaggtggcttgggctcacctggcaatcggacaaccaaggagaagaaggtgtttatcagtctagtgggctctcggggcctgggctgcagcatctccagtggccccatccagaagcctggcatcttcgtcagccacgtgaagcctggctccctgtctgcagaggtggggttagagacaggagaccagattgtggaagtcaatggcatagacttcaccaacctggaccacaaggaggctgtgaatgtcctgaagagcagccgcagcctgaccatctccatcgttgctggagccggccgggagctgttcatgacggaccgggaacggctggaggaggcacggcagcgtgagctgcaacggcaggaactcctcatgcagaagcggctggccatggagtccaacaagatcctccaggagcagcaggagatggagcgccagaggagaaaggagatcgcccagaaggctgccgaggagaatgagagataccggaaggagatggaacagatctcggaggaggaagag

SEQ ID NO: 6 - DNA, 399 bp, Human
ggcagacagcgttgggcttcaggtaggggatgaaattgtccggatcaacggctattccatctcttcctgtacccatgaggaagtcatcaacctgatccgcaccaagaagaccgtgtccatcaaagtgagacacatcggactgatccctgtgaagagctctcctgaggagtccctcaaatggcagtatgtggatcagttcgtgtcggaatctgggggtgtgcgaggtggcttgggctcacctggcaatcggacaaccaaggagaagaaggtgtttatcagtctagtgggctctcggggcctgggctgcagcatctccagtggccccatccagaagcctggcatcttcgtcagccacgtgaagcctggctccctgtctgcagaggtggggttagagacagg

SEQ ID NO: 7 - protein, 279 aa, Human
  Translation of SEQ ID No5
LDRLHPEGLGLSVRGGLEFGCGLFISHLIKGGQADSVGLQVGDEIVRINGYSISSCTHEEVINLIRTKKTVSIKVRHIGLIPVKSSPEESLKWQYVDQFVSESGGVRGGLGSPGNRTTKEKKVFISLVGSRGLGCSISSGPIQKPGIFVSHVKPGSLSAEVGLETGDQIVEVNGIDFTNLDHKEAVNVLKSSRSLTISIVAGAGRELFMTDRERLEEARQRELQRQELLMQKRLAMESNKILQEQQEMERQRRKEIAQKAAEENERYRKEMEQISEEEE

SEQ ID NO: 8 - protein, 132 aa, Human
  Translation of SEQ ID No6
ADSVGLQVGDEIVRINGYSISSCTHEEVINLIRTKKTVSIKVRHIGLIPVKSSPEESLKWQYVDQFVSESGGVRGGLGSPGNRTTKEKKVFISLVGSRGLGCSISSGPIQKPGIFVSHVKPGSLSAEVGLET